(19) United States
(12) Patent Application Publication
(10) Pub. No.: US 2023/0201499 A1
(43) Pub. Date: Jun. 29, 2023

(54) PISTON RESUSCITATOR AND/OR VENTILATOR SYSTEMS, DEVICES, AND METHODS FOR USING SAME

(71) Applicant: Respirana, Inc., San Ramon, CA (US)

(72) Inventors: Dylan DeLonzor, Lakewood, CO (US); Russell DELONZER, San Ramon, CA (US); Neil Padharia RAY, Sacramento, CA (US); Jessica Brown, Lakewood, CO (US)

(21) Appl. No.: 17/915,768

(22) PCT Filed: Apr. 5, 2021
(86) PCT No.: PCT/US2021/025814

§ 371 Date: Sep. 29, 2022

Related U.S. Application Data
(60) Provisional application No. 63/005,435, filed on Apr. 5, 2020.

(51) Int. Cl. A61M 16/00; A61M 39/10; A61M 16/10; A61M 16/20

(52) U.S. Cl. CPC A61M 16/0072; A61M 16/10; A61M 16/022; A61M 16/0051; A61M 16/0084; A61M 16/208; A61M 39/10

(57) ABSTRACT

Resuscitation/ventilation systems that include a pressure chamber or cylinder may use a piston articulated within the pressure chamber or shaft to push air and/or a mixture of gas and air into and out of an airway circuit for the purpose of providing mechanical ventilation and/or artificial respiration to a patient. In some cases, the pressure chamber or cylinder may be resident within a canister that fits with a body. The canister may include a motor that moves a shaft connected to the piston up and down, or in and out, within the pressure chamber or cylinder and this movement of the piston may cause a vacuum within the airway circuit and/or the pushing of air or gas out of the airway circuit into a patient’s lung(s).

### RELATED APPLICATION

This patent application is an INTERNATIONAL/PCT application claiming priority to U.S. Provisional Pat. Application No. 63/005,435, filed on 05 Apr. 2020 and entitled “PISTON RESUSCITATOR AND/OR VENTILATOR SYSTEMS, DEVICES, AND METHODS FOR USING SAME,” which is incorporated in its entirety herein.

### FIELD OF INVENTION

The present invention is in the field of medical devices and, more particularly, in the field of resuscitators and ventilators.

### BACKGROUND

Traditional ventilators are large and expensive pieces of equipment that require specialized training to use. Because of their size and the training required to operate them, it can be difficult to use a traditional ventilator outside of a hospital, particularly out of an intensive care unit of a hospital which leaves many patients in situations outside of a hospital or intensive care unit (e.g., an ambulance or assisted care facility) without the devices they need to breath in order to sustain life and/or recover from an illness. In addition, the financial cost of ventilators prevents their use in many situations.

### SUMMARY

Disclosed herein are exemplary resuscitation/ventilation systems and airway circuits coupled to the resuscitation/ventilation systems that may facilitate the ventilation and/or provision of artificial respiration to a patient on a temporary (e.g., 30 minutes, 2 hours, etc.) or more permanent basis (e.g., days, weeks, or months). In some embodiments, the resuscitation/ventilation system has two primary components, a canister and a body. The body may house the canister and may provide controls or instructions to the canister that govern its operation (e.g., the provision of air and gases to the patient via an airway circuit). The canister may be configured to be removable from the body and, in some instances, may operate outside of the body. In some cases, the canister may be interchangeable with another similarly configured canister.

Exemplary canisters may be configured to be removably inserted into an opening in a body. The canisters may include a hollow cylindrical chamber configured to hold a volume of gas that may be pushed through the airway circuit to the patient via an opening in the hollow cylindrical chamber through which gas may pass into a respiratory circuit for a patient as a piston positioned within the hollow cylindrica chamber translates between the first and second positions. Translation of the piston between the first and second positions and vice versa may be facilitated by a motor (e.g., a stepper motor) configured to move the piston from the first position to the second position and subsequently move the piston from the second position to the first position within the hollow cylindrical chamber. In some instances the piston may be coupled to a shaft that is in communication with the motor and, in these embodiments, movement of piston may be facilitated by the motor moving the shaft.

The body may also include a first communication/power interface coupling portion that may be configured to couple to a corresponding second communication/power interface coupling portion that is communicatively and electrically coupled to the body and establish communicative and electrical coupling between the canister and the body.

The body may include the opening configured for acceptance of a portion of the canister therein, a controller configured to control the motor, and the second communication/power interface coupling portion configured to couple to the first communication/power interface coupling portion and establish communicative and electrical coupling between the canister and the body. The body may also include a power source configured to provide electrical power to the body and the cannister.

Movement of the piston from the first position to the second position and vice versa may be responsive to a communication from the controller that is communicated to the motor via a coupling of the first and second communication/power interface coupling portions. In some embodiments, body may further include a cord that is coupled to the second communication/power interface coupling portion. The cord may be any length, which may enable the canister to operate away from and/or outside of the body while still being in electrical and communicative communication with the body.

In some embodiments, the body may further include a control panel via which a user (e.g., a clinician or respiratory therapist) may provide one or more settings for the operation of the resuscitation/ventilation system . Exemplary settings include, but are not limited to, a maximum pressure, an inhale to exhale (I/E) ratio, a number of breaths per minute, and a volume of gas delivered to a patient.

In some embodiments, the body may also include a motor driver communicatively coupled to the controller and configured to provide instructions to the motor that cause the motor to move the piston between the first position and the second position and between the second position and the first position.

### BRIEF DESCRIPTION OF THE FIGURES

The present invention is illustrated by way of example, and not limitation, in the figures of the accompanying drawings in which:

FIG. 1A provides a view of an in-line piston resuscitator system in a first state of operation, consistent with some embodiments of the present invention;

FIG. 1B provides a view of the in-line piston resuscitator system in a second state of operation, consistent with some embodiments of the present invention;

FIG. 2 is a block diagram of another exemplary piston resuscitator and/or ventilator system, consistent with some embodiments of the present invention;

FIG. 3 is a block diagram of another exemplary piston resuscitator and/or ventilator system, consistent with some embodiments of the present invention;

FIG. 4A provides a front plan view of an exemplary resuscitation/ventilation system , consistent with some embodiments of the present invention;

FIG. 4B provides a side plan view of the resuscitation/ventilation system of FIG. 4A, consistent with some embodiments of the present invention;

FIG. 4C provides a front plan exploded view of the resuscitation/ventilation system of FIG. 4A, consistent with some embodiments of the present invention;

FIG. 4D provides a top perspective view of a body of the resuscitation/ventilation system of FIG. 4A, consistent with some embodiments of the present invention;

FIG. 4E provides a top plan view of the exemplary body of FIG. 4D, consistent with some embodiments of the present invention;

FIG. 4F provides a back plan view of the exemplary body of FIG. 4D, consistent with some embodiments of the present invention;

FIG. 5A shows a side plan view of an exemplary canister configured to fit within and cooperate with the body of FIG. 4D, consistent with some embodiments of the present invention;

FIG. 5B provides a bottom perspective view of the exemplary canister of FIG. 5A, consistent with some embodiments of the present invention;

FIG. 5C provides a cross-section view of the exemplary canister of FIG. 5A, consistent with some embodiments of the present invention;

FIG. 6A is a block diagram showing exemplary components of an airway circuit, consistent with some embodiments of the present invention;

FIG. 6B shows an air pathway during an inhalation stroke for the airway circuit of FIG. 6A, consistent with some embodiments of the present invention;

FIG. 6C shows an air pathway during an exhalation stroke for the airway circuit of FIG. 6A, consistent with some embodiments of the present invention;

FIG. 7 is a block diagram showing exemplary components of a system in which computer readable instructions instantiating the methods of the present invention may be stored and executed, consistent with some embodiments of the present invention;

FIG. 8 is a flowchart showing a process for preparing and providing instructions to a resuscitation/ventilation system for the provision ventilation to a patient, in accordance with some embodiments of the present invention;

FIG. 9 is a flowchart showing a process for executing instructions to operate a resuscitation/ventilation system for the provision ventilation and/or artificial respiration to a patient, in accordance with some embodiments of the present invention;

FIG. 10A provides a first graphic user interface (GUI) that shows various settings and performance metrics for a resuscitation/ventilation system and/or components thereof, in accordance with some embodiments of the present invention;

FIG. 10B provides a second GUI that shows various settings and performance metrics for the resuscitation/ventilation system and/or components thereof, in accordance with some embodiments of the present invention; and

FIG. 10C provides a third GUI that shows various settings and performance metrics for the resuscitation/ventilation system and/or components thereof, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Disclosed herein are piston resuscitator and/or ventilator systems and devices and methods for using the resuscitator and/or ventilator systems and devices. The in-line piston resuscitator and/or ventilator system cyclically transitions between a first state where the resuscitator and/or ventilator system assists a patient with the expiration of air and carbon dioxide from the lungs (i.e., exhalation) to a second state where the resuscitator and/or ventilator system provides the patient’s lungs with air (which may be supplemented with oxygen), which may correspond to a patient’s inhalation. The cyclical transitions between the first and second states of the exemplary in-line piston resuscitator and/or ventilation system may correspond with an inhale-to-exhale ratio (which may be represented herein as “I:E”) for the patient that may be proscribed by an attending physician or other medical professional depending on the patient’s needs and state of health.

The piston resuscitator and/or ventilator systems disclosed herein may be configured to facilitate user/treatment provided control of the air/gas pressure during an inhalation phase and an exhalation phase for the piston resuscitator and/or ventilator systems, a waveform shape or other timing for the cycle of inhalation and exhalation (e.g., frequency and/or duration of time for the inhalation phase and exhalation phase), and/or breaths per minute.

In some instances, the piston resuscitator and/or ventilator systems disclosed herein may use one or more commonly known/used manual resuscitator devices (bag mask ventilation (BVM)) equipment and ventilator bags. Alternatively, one or more of the piston resuscitator and/or ventilator systems disclosed herein may not use a ventilator or oxygen reservoir and may be, for example, directly coupled to a source of air/gas.

The resuscitator and/or ventilator systems disclosed herein may be used/indicated as an Emergency Use Resuscitator System (EURS) to supply breathing gasses to intubated or tracheotomized patients in acute respiratory distress who are not spontaneously breathing. In some cases, the resuscitator and/or ventilator systems disclosed herein may be used adjunctively with cleared adult manual resuscitators in emergency situations when traditional critical care equipment is not available. Exemplary patients that may use the resuscitator and/or ventilator system described herein include intubated adult patients with body weight greater than 30 kg (66 pounds) experiencing respiratory failure in emergency situations when a critical care ventilator is not available.

In some embodiments, the resuscitator and/or ventilator systems disclosed herein may be used to facilitate ventilation during inter-hospital transport and/or to provide uninterrupted ventilation/breathing support while a patient undergoes a procedure or test (e.g., surgery or an MRI) outside of the critical care unit/ICU. In some instances, transportability of a patient requiring ventilation may be facilitated by having a first portion (e.g., a smaller or lighter portion and/or the portion of the resuscitator and/or ventilator systems that provides air/gas for the patient) of the resuscitator and/or ventilator systems disclosed herein on a stretcher or a lap of a patient in a wheelchair and a second portion (e.g., a base, or controller unit) that provides, for example, power or operation instructions to the first portion that is physically closer to the patient and/or the patient’s mouth. The second portion may, for example, be coupled to a pole extending from the stretcher and/or wheelchair and/or extending from a base (typically on wheels) that is moved along with the stretcher/wheelchair when the patient is moved and/or temporarily positioned in, for example, an ambulance, a hallway and/or a procedure room.

In some embodiments, resuscitator and/or ventilator systems disclosed herein may be used in place of manual resuscitation and/or in situations where manual resuscitation is a standard of care such as with emergency response to a cardiac and/or respiratory event.

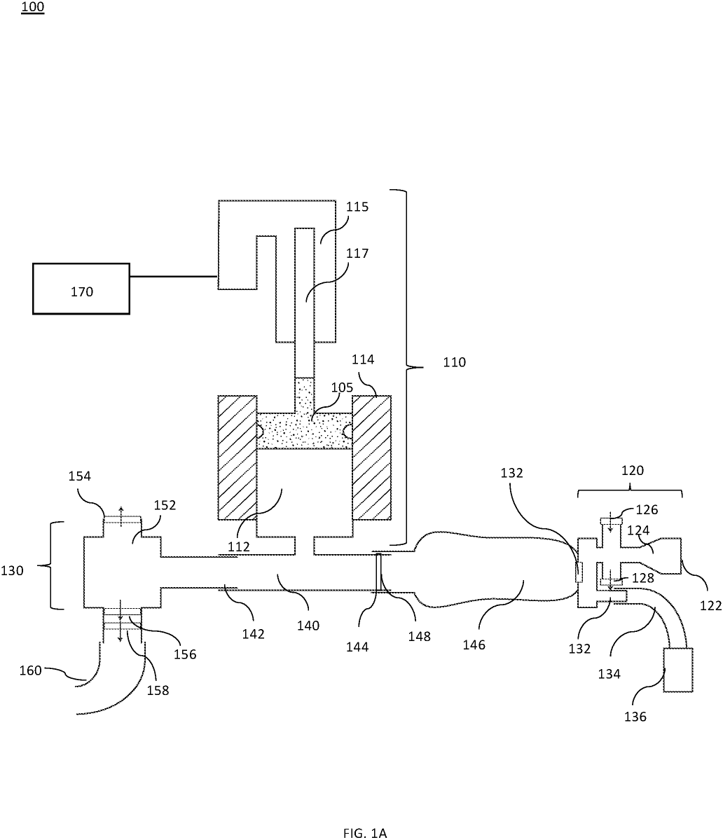

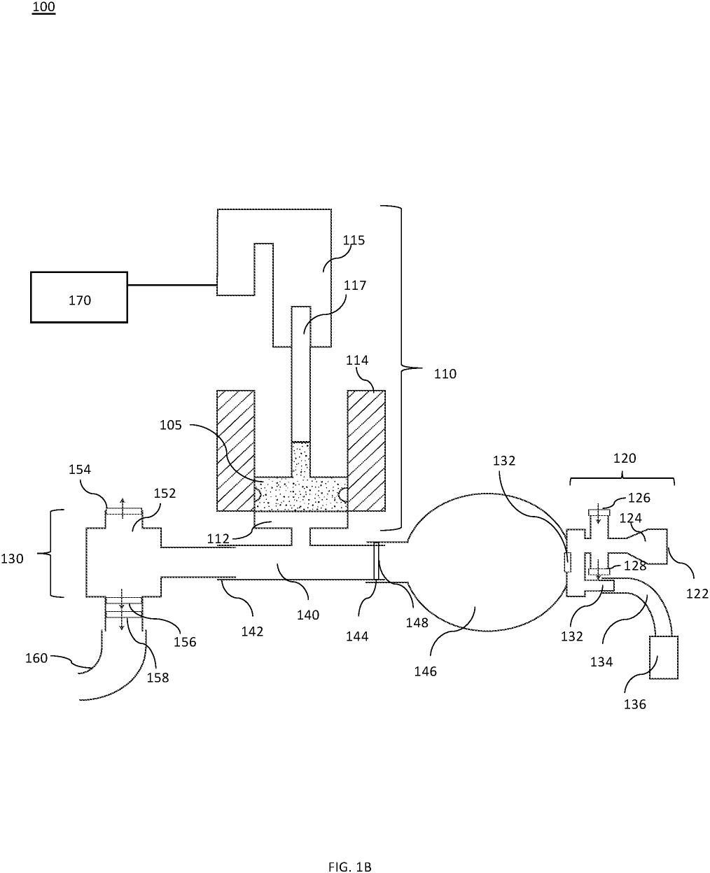

FIG. 1A provides a view of an exemplary in-line piston resuscitator and/or ventilator system 100 in the first state of operation, or first position, and FIG. 1B provides a view of the exemplary in-line piston resuscitator and/or ventilator system 100 in the second state of operation, or in a second position. Ventilation system 100 comprises a piston assembly 110, an air/gas intake assembly 120, an air/gas exhaust assembly 130, and a control panel 170. Control panel 170 may be configured to provide information to and/or receive instructions from a user or patient regarding the operation of ventilation system 100 via a user interface (not shown) like a display, keypad, dials, or touch screen display. Control panel 170 may include a processor or other components configured to operate and/or control the operation of one or more components of ventilation system 100 and, in particular, the operation of piston assembly 110.

Piston assembly 110 includes a linear actuator 115 that is configured to linearly move rod 117 that is coupled to a piston 105 so that piston 105 moves linearly within a chamber, or lumen, 112 of a cylinder 114 from a first state as shown in FIG. 1A to a second state as shown in FIG. 1B. Piston 105 and cylinder 114 may be configured so that piston 105 completely (or nearly occupies) the area of chamber 112 thereby forming an air-tight, or nearly air-tight seal. In some embodiments, a lubricant (not shown) may be present at the interface between piston 105 and cylinder 114 within chamber 112. The lubricant may be configured to, for example, reduce friction between piston 105 and cylinder 114 while piston 105 is transitioning between the first state and the second state and/or form an air-tight, or nearly air-tight seal between piston 105 and cylinder 114.

Linear actuator 115 may be configured to cyclically move a rod 117 included therein that is coupled to piston 105 according to an I:E ratio for a patient who is using ventilation system 100 so that piston 105 moves within chamber 112 in a manner that corresponds to the I:E ratio. The I:E ratio for the linear actuator may be set manually via, for example, manipulation of a dial or lever in communication with linear actuator 115 and/or electronically via control panel 170.

In some embodiments, linear actuator 115 may include and/or be coupled to a feedback mechanism that provides linear actuator 115 with information regarding the position, or state, of rod 117 and thereby piston 105. Exemplary feedback mechanisms include, but are not limited to, optical sensors (not shown) and potentiometers (not shown). Exemplary linear actuators 115 include the optical feedback linear actuator and the feedback rod actuator both of which are sold/distributed by FIRGELLI AUTOMATIONS™.

Cylinder 114 and piston 105 may be a component that is pre-assembled and/or designed to work together such as the FLAT-1 pneumatic cylinder and/or standard TA series NFPA cylinder manufactured and/or distributed by BIMBA™. Rod 117 may be coupled to the top of piston 105 via any appropriate method including, but not limited to, chemical, mechanical, ultrasonic, and heat bonding.

Cylinder 114 and/or chamber 112 may be coupled to an adaptor 140 configured to couple to cylinder 114 and/or chamber 112 to air/gas exhaust assembly 130 via a first adaptor coupling 142 and couple to cylinder 114 and/or chamber 112 to a ventilation bag 146 via a second adaptor coupling as shown in FIGS. 1A and 1B. Cylinder 114 and/or chamber 112 may be coupled to an adaptor 140 via, for example, a rigid and/or flexible tube that may resemble tubes commonly used in ventilator circuits. In some embodiments, an optional flapper valve 148 may be positioned within adaptor 140 or ventilation bag 146 and/or a coupling therebetween 144. Flapper valve 148 may be positioned and/or configured to prevent backflow of air/gas into ventilation bag 146 when, for example, piston 105 is in the second state and/or is transitioning from the first state to the second state. Ventilation bag 146 may be, for example, a self-inflating bag such as an AMBU™ bag and/or a respirator bag. Air/gas intake assembly 120 includes an air/gas intake assembly 122 that includes an air/gas/oxygen reservoir bag 124, a one-way intake valve 126, a one-way exhaust valve 128, and an oxygen line coupling/port 132 that is coupled to an oxygen line 134 which is coupled to an oxygen supply 136 (e.g., cannister of compressed oxygen gas). Air/gas intake assembly 122 is coupled to a ventilation bag 146 via a coupling 132 that includes a one-way valve.

Air/gas exhaust assembly 130 includes an air/gas exhaust manifold 152 which includes a one-way exhaust valve 154, a one-way valve 156, and an optional one-way exhaust valve 158 that couples to a respirator tube 160 that is configured to be coupled to a patient interface (not shown) that operates to push air/gas received from air/gas intake assembly 120 down respirator tube 160 to be delivered to the patient and also exhausts air/gas exhaled from the patient’s lungs via air/gas exhaust assembly 130. Exemplary patient interfaces include, but are not limited to, face masks, laryngeal mask airways, and endotracheal tubes. Respirator tube 160 may be of any appropriate length to accommodate positioning of the patient interface on/in the patient and coupling of respirator tube 160 to system 100. For example, if system 100 is placed directly next to a patient (e.g., in bed with the patient) then a length of respirator tube 160 may be relatively short (e.g., 6-18 inches). Alternatively, if system 100 is placed not as closely to the patient as may be the case when system 100 is placed on, for example, a bedside table or pole then respirator tube 160 may be relatively long (e.g., 2-8 feet). Optionally, the patient interface and/or air/gas exhaust assembly 130 may include a positive end-expiratory pressure (PEEP) valve (not shown). Additionally, or alternatively, some embodiments, of system 100 may include a manometer and/or an over-pressure pop off valve to manage PEEP and/or other pressure values within system 100 and/or the patient. Additionally, or alternatively, some embodiments, of system 100 may include a pressure sensor and/or a flow sensor that may be configured to sense and/or measure air/gas pressure and air flow rates, respectively within system 100 and/or the patient.

TABLE 1

| | | | | |
|---|---|---|---|---|
| Pressure Vessel (Squared) | Volume First State | Volume Second State fa | Air Pressure First State | Air Pressure Second State |
| Piston assembly 110 | Positive | Negative | Negative | Positive |
| Air/gas intake assembly 122 | N/A | N/A | Negative | Positive |
| Ventilation bag 146 | Negative | Positive | Negative | Positive |
| Air/gas/oxygen reservoir bag 124 | Negative | N/A | Negative | N/A |
| Air/gas exhaust assembly 130 | N/A | N/A | Negative | Positive |
| Respirator tube 160 | N/A | N/A | N/A | Positive |

Table 1 provides a summary of the volume (positive or negative) and air pressure (positive or negative) for various parts of system 100 during the first state shown in FIG. 1A and the second state shown in FIG. 1B. When system 100 is in its first state, piston 105 is positioned in an upper portion of chamber 112 so that a volume of chamber 112 is positive and air/gas within lumen is not compressed so that the air pressure within chamber 112 is negative. Air/gas intake assembly 122 does not change in volume when system 100 transitions between the first and second states. However, when system 100 is in the first state, the air pressure within air/gas intake assembly 122 is negative. The volumes of ventilation bag 146 and air/gas/oxygen reservoir bag 124 are negative and the air pressure in ventilation bag 146 and air/gas/oxygen reservoir bag 124 is negative when system 100 is in the first state. A volume of air/gas exhaust assembly 130 and the respirator tube 160 does not change when system 100 translates between the first and second state. However, when system 100 is in the first state, air/gas exhaust assembly 130 has negative air pressure.

The air/gas in chamber 112 while system 100 is in the first state may be provided by ventilation bag 146 and air/gas intake assembly 120 via adaptor 140. When the linear actuator 115 pushes piston 105 down within chamber 112 to transition system 100 from the first state to the second state, air/gas within chamber 112 is compressed (positive pressure) so that the volume of chamber 112 decreases. The compressed air/gas is then forced into adaptor 140 where it may be delivered to air/gas exhaust assembly 130 thereby creating a positive pressure in air/gas exhaust assembly 130. The air/gas is then pushed via one-way valve 156 into respirator tube 160 thereby providing a positive air/gas pressure in respirator tube 160. The air/gas in respirator tube 160 is then delivered to the patient interface so that it may be provided to the patient’s lungs.

When transitioning back to the first state, linear actuator 115 may pull rod 117 upwards thereby pulling piston 105 upwards to create a positive volume in chamber 112 and negative pressure in ventilation bag 146 which may open a one-way valve positioned within coupling 132 to create a negative pressure in air/gas intake assembly 122 and/or oxygen bag 124. This negative air pressure in air/gas intake assembly 122 and oxygen bag 124 may cause ambient air to be pulled into air/gas intake assembly 122 via one-way intake valve 126 and/or oxygen bag 124. In some embodiments, the air pulled into air/gas intake assembly 122 may be oxygenated via oxygen provided by oxygen supply 136 by way of oxygen line 134. Exhaust valve 128 may be configured to be a pressure release valve.

A frequency, or timing for, transitioning between the first and second states may be set by an inhale-to-exhale (I:E) ratio or a duration of time (e.g., 1 s inhale, 2 s exhale or 2 s inhale 4 s exhale) for a patient’s inhalation and exhalation where transitioning from the first state to the second state represents a patient’s inhalation cycle and transitioning from the second state to the first state represents the patient’s exhalation cycle. The frequency of transitioning between the first and second states may be set by, for example, a physician or medical professional via interaction with control panel 170.

The valves disclosed herein may be pre-approved by a governmental agency (e.g., Food and Drug Administration (FDA)) for use in a clinical setting and/or with a ventilator. In some embodiments, system 100 may be designed to be partially and/or wholly disposable, which may eliminate the need for cleaning or servicing of system 100 thereby conserving these resources. In the event of a power outage and/or failure of an electronically driven portion of system 100 (e.g., control pad 170 and/or linear actuator 115), system 100 may be manually operated by, for example, manually squeezing ventilation bag 146.

In some embodiments, one or more components of system 100 may be resident within a housing (not shown). For example, control panel 170 and piston assembly 110 may be resident within a housing. The housing may be configured to be coupled to a mounting and/or stability device such as a pole or shelf. In some examples, the housing may rest in a bed with a patient.

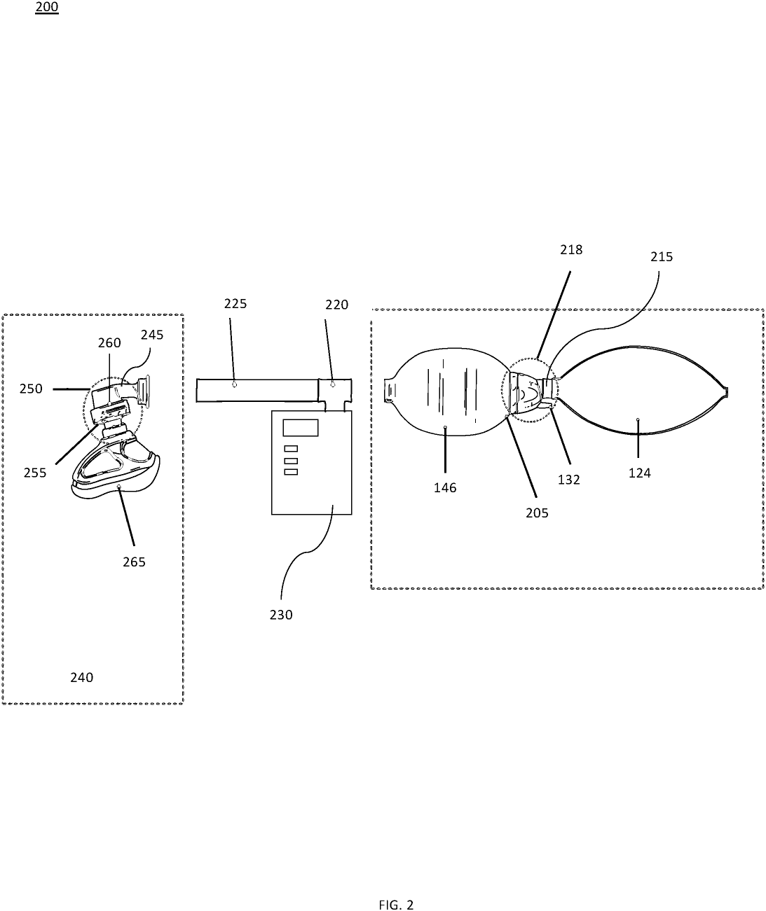

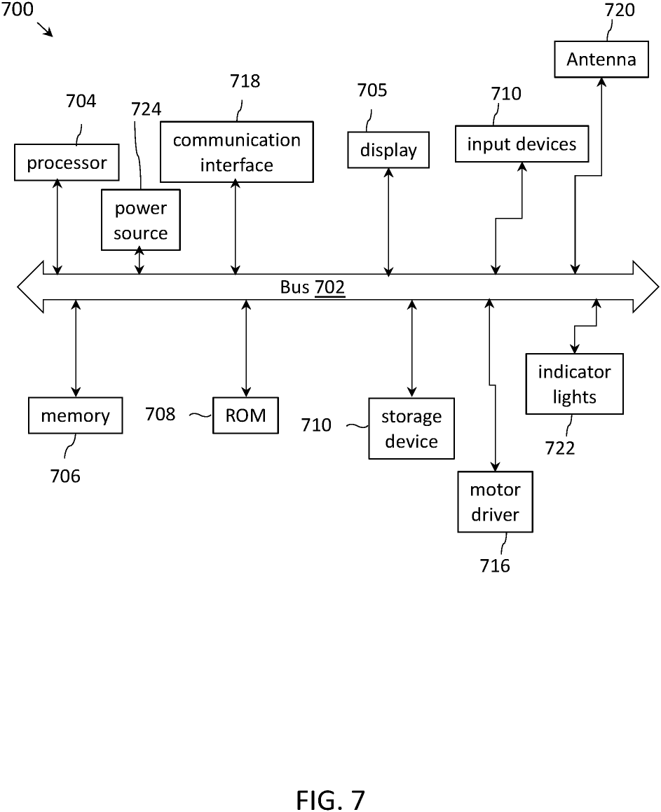

FIG. 2 is a block diagram of an exemplary piston resuscitator and/or ventilator system 200 that includes an air/gas intake assembly 210, a patient Interface assembly 240, and a control/piston system 230. Air/gas intake assembly 210 includes an air/gas intake manifold 215 that is coupled to air/gas/oxygen reservoir bag 124 and a ventilation bag 146 via a coupling 132. The air/gas intake assembly 210 includes a one-way intake valve 218 and oxygen line coupling/port 132 that may be coupled to an oxygen line like oxygen line 134 (not shown). Ventilation bag 146 is coupled to a tee 220 which is coupled to control/piston system 230 and a tube 225 connecting to patient Interface assembly 240. In some embodiments, tee 220 may include a pop-off valve (not shown). Tube 225 may be any appropriate length and is coupled to a patient manifold 245. Patient manifold 245 includes a lip valve 260, a patient valve 250, a disk membrane 255 and a coupling to a patient mask 265. System 200 may be configured to operate in a manner similar to the operation of system 100 in that system 200 may ventilate a patient through movement of a piston housed within control/piston system 230. Further details regarding the components and operation of control/piston system 230 are provided below with regard to FIG. 7. Although system 200 shows the patient manifold 245 being coupled to a mask (i.e., patient mask 265), patient manifold 245 may be configured to be coupled to another apparatus configured to facilitate an air/gas exchange with a patient’s lungs such as an endotracheal tube or a tracheostomy tube.

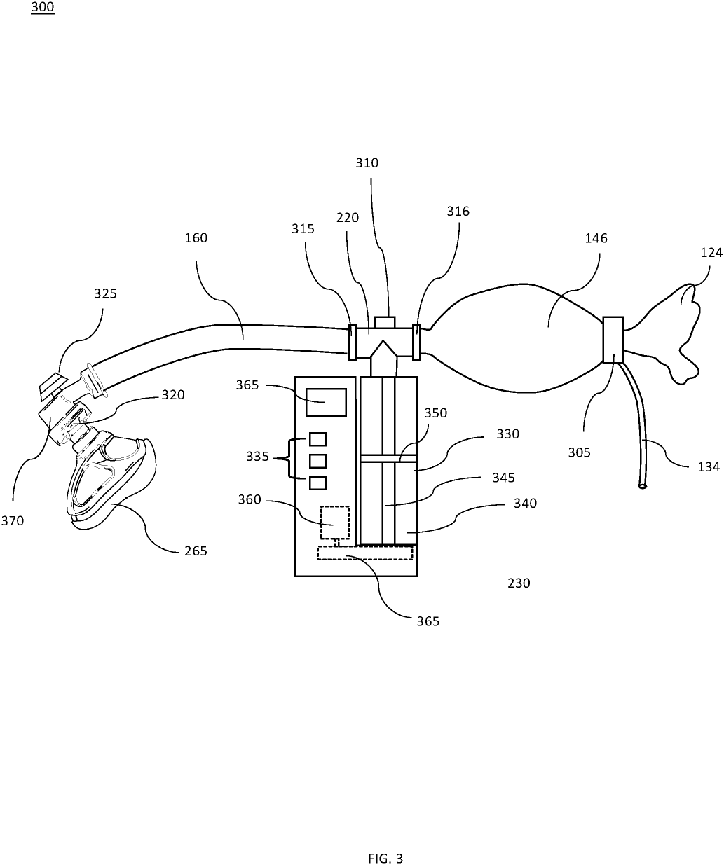

FIG. 3 is a block diagram of an exemplary piston resuscitator and/or ventilator system 300. System 300 may also be referred to as an airway circuit 300 that includes a piston resuscitation/ventilation system 230. System 300 includes air/gas/oxygen reservoir bag 124 coupled to ventilation bag 146 via a coupling or manifold that includes an intake valve 305 and an oxygen port coupled to an oxygen line 134. Ventilation bag 146 may be coupled to tee 220 (or other coupling) which may include a pop-off valve 310 and a one-way valve 316 may be positioned between ventilation bag 146 and tee 220. Tee 220 may be physically and/or pneumatically coupled to a cylinder, or chamber, 330 of control/piston system 230. Cylinder 330 may include a chamber that houses a shaft 345 (e.g., a drive screw) coupled to a piston 350. Shaft 345 may be coupled to a piston drive 355 which may include a motor 360 (e.g., a stepper motor). Motor 360 and piston drive 355 may be configured to move piston 350 from a first state or position to a second state or position (e.g., up and down) within a cylinder 330 in a manner similar to the movement of piston 105. Control/piston system 230 may include a control panel 335 that may be similar to and/or include control panel 170 by which a user may input instructions and/or set one or more parameters for the operation of system 300. Control/piston system 230 may also include a display device 365 (e.g., LCD or LED display) which may be configured to provide information to a user regarding, for example, the operation of control/piston system 230, an amount of air pressure in a portion of system 200, and/or present alarm notifications.

Tee 220 may be coupled to a length of respirator tube 160 and a one way valve 315 may be positioned at, or near, the joint between tee 220 and respirator tube 160. Respirator tube 160 may be coupled to a patient manifold 370, which may be similar to patient manifold 130 and/or 245. Patient manifold 370 may include a PEEP valve 325 a patient valve 320 and a patient mask 265 configured to facilitate an air/gas exchange with a patient’s lungs. In some embodiments, PEEP valve 325 may be positioned further away from the patient interface and/or patient manifold 370 than shown but will may be positioned prior to the 1-way valve. Although system 300 shows the patient manifold 370 being coupled to a mask (i.e., patient mask 265), patient manifold 370 may be configured to be coupled to another apparatus configured to facilitate an air/gas exchange with a patient’s lungs such as an endotracheal tube or a tracheostomy tube.

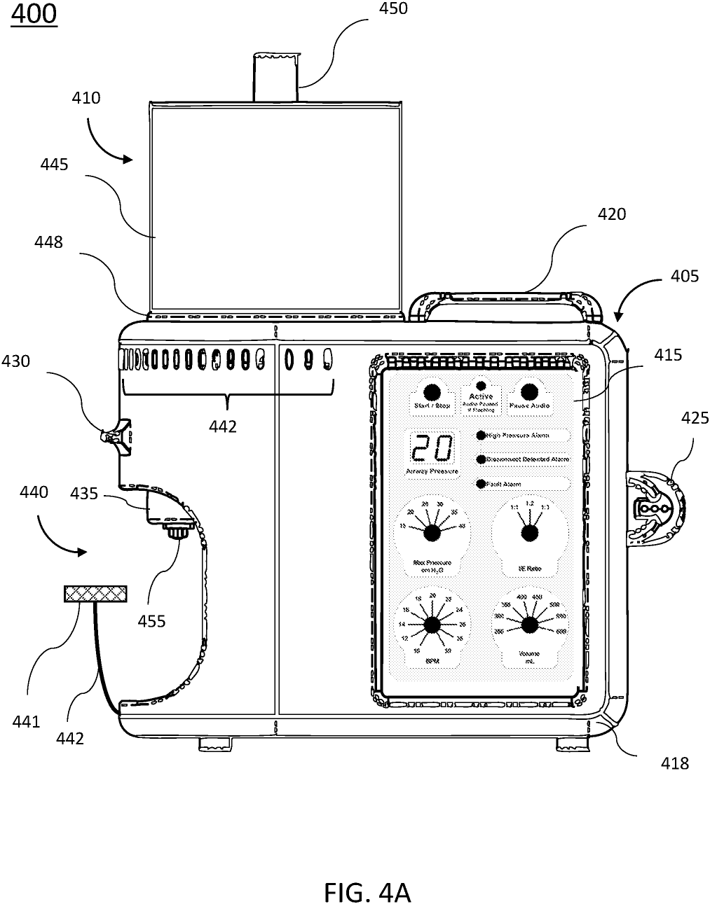

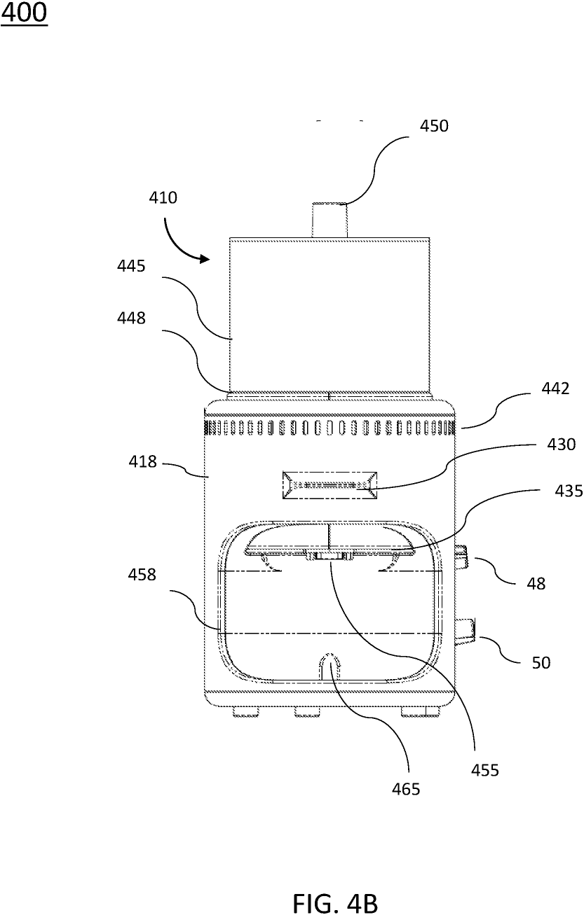

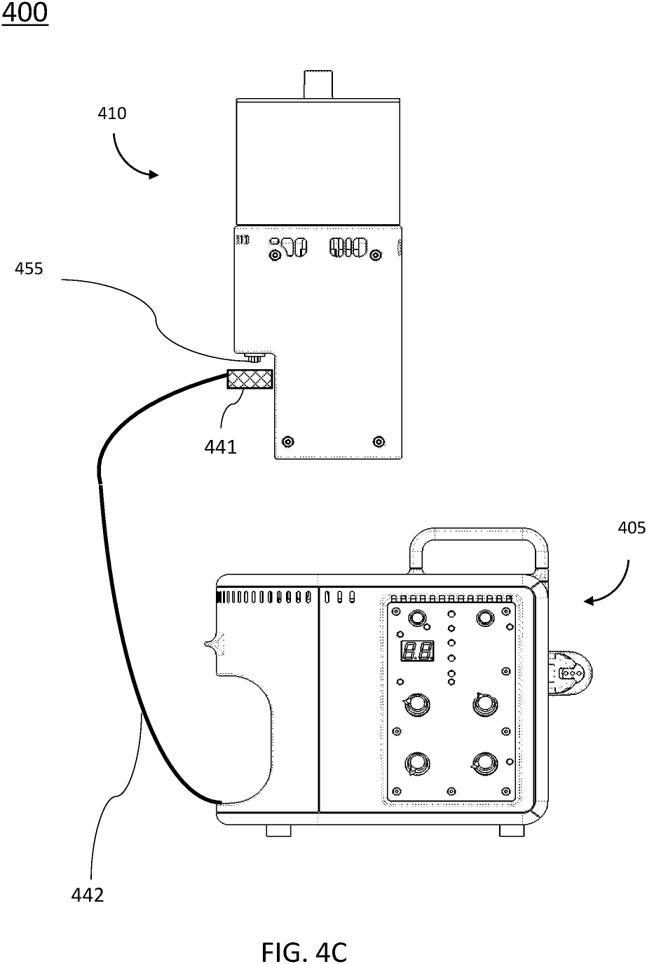

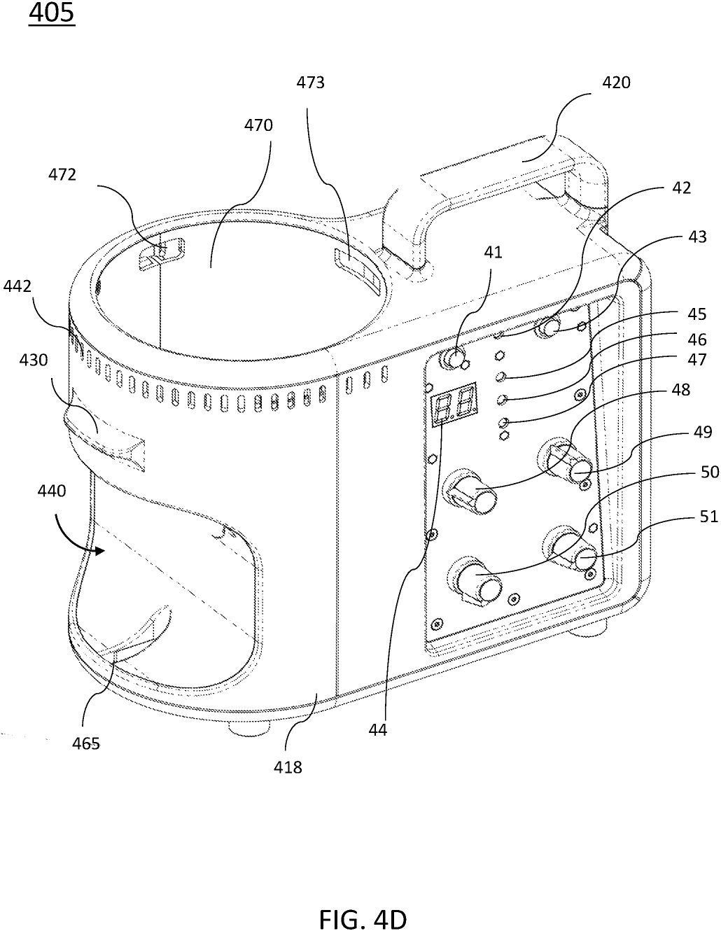

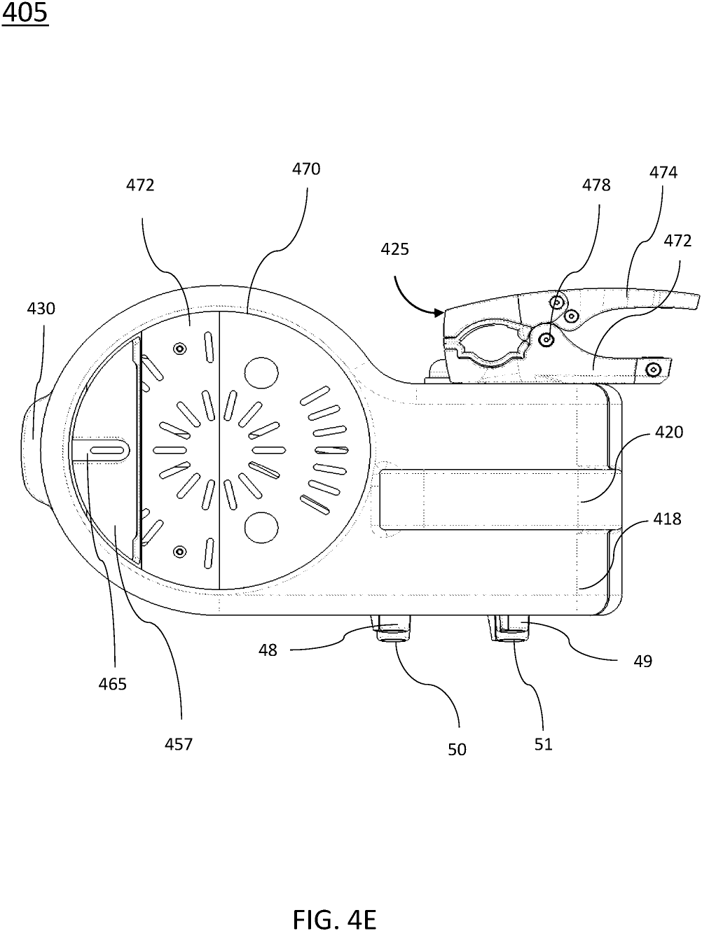

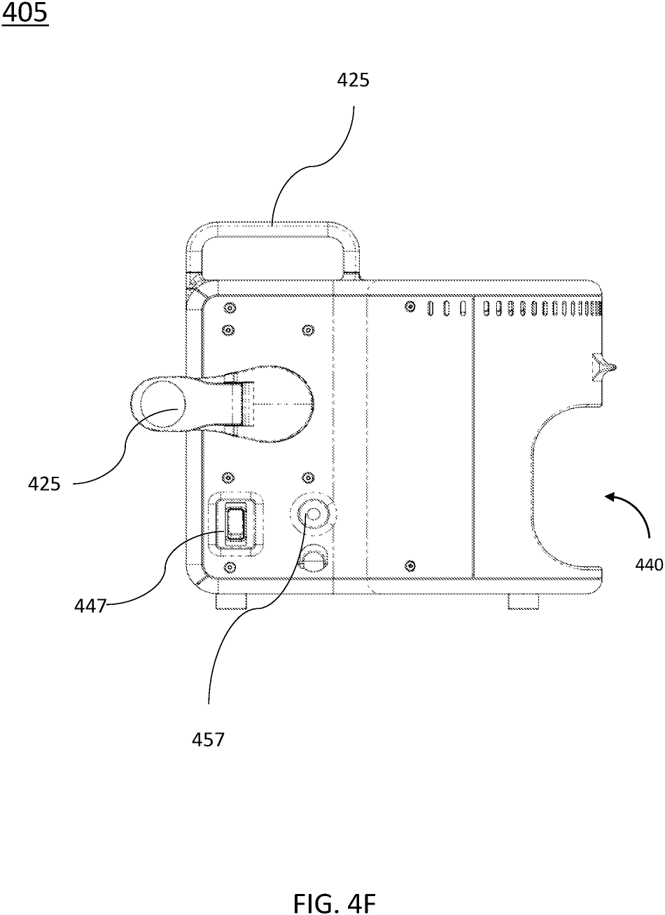

FIGS. 4A-4D provide various views of an exemplary piston-driven resuscitation/ventilation system 400, which may, in some instances, be similar to control/piston system 230. In particular, FIG. 4A provides a front plan view of resuscitation/ventilation system 400, FIG. 4B provides a side plan view of resuscitation/ventilation system 400, FIG. 4C provides a back plan view of resuscitation/ventilation system 400, and FIG. 4D provides a front plan exploded view of resuscitation/ventilation system 400.

As shown in FIG. 4A, resuscitation/ventilation system 400 includes a body 405 and a canister 410 resident within body 405. Body 405 includes a control panel 415, a housing 418, a handle 420, a mounting mechanism 425, an engagement mechanism 430, a first electrical and/or communication coupling portion 455, an opening 440, a set of ventilation openings 442, and a canister joint 448. The portion of canister 410 showing in FIG. 4A (i.e., the portion of canister 410 not positioned within/housed by body 405) includes a hollow cylindrical chamber 445, an extension 450 configured to couple to one or more components (e.g., tubes or joints (e.g., elbows, straight line couplings, T-connectors, and/or Y-connectors) that may facilitate air/gas exchange with a patient being resuscitated and/or ventilated by resuscitation/ventilation system 400, and an upper edge of an indented portion of canister 435 that includes first electrical and/or communication coupling portion 455. Both upper edge of an indented portion of canister 435 and first electrical and/or communication coupling portion 455 may be seen through opening 440. Ventilation openings 442 may serve to provide ambient air to internal components of body 405 in order to, for example, cool these internal components.

Resuscitation/ventilation system 400 may be made from any suitable materials including, by not limited to, plastic, metal, latex, and/or composites. Overall dimensions for body 405 range from, for example, 6-14 inches in length, 3-9 inches in width, and 4-16 inches in height. Handle 420 may be configured to facilitate the hand carrying of body 405 and/or resuscitation/ventilation system 400.

First electrical and/or communication coupling portion 455 may be configured to communicatively and electrically couple to a corresponding second electrical and/or communication coupling portion (not shown). In some cases, first electrical and/or communication coupling portion 455 may be configured to mechanically couple with the second electrical and/or communication coupling portion via, for example, one or more screws, clips, and/or friction.

First electrical and/or communication coupling portion 455 may be configured to receive instructions regarding an operation of one or more components of canister 410 (e.g., a motor) from a processor and/or controller (not shown) resident within body 405. Additionally, or alternatively, first electrical and/or communication coupling portion 455 may be configured to draw electrical power from body 405 the second electrical and/or communication coupling portion so, for example, execute received instructions.

Control panel 415 includes a start/stop button 482, an active indicator light 484, a pause audio button 486, an airway pressure indicator window 488, a high pressure alarm indicator light 490, a disconnect detected alarm indicator light 492, a fault alarm indicator light 494, a maximum pressure in centimeters of water H2O dial 496, an I/E ratio dial 498, a breaths-per-minute (BPM) dial 499, and a volume in milliliters (mL) dial 500. Ventilation and/or resuscitation delivered by resuscitation/ventilation system 400 may be initiated and/or paused via activation of start/stop button 482. A maximum pressure may correspond to a maximum airway pressure permitted in an airway circuit such as the airway circuits disclosed herein. Volume dial 500 may be used to adjust a tidal volume within an airway circuit.

Active indicator light 484 may be configured to be lit when resuscitation/ventilation system 400 is on and/or providing ventilation and/or resuscitation to patient. Pause audio button 486 may be configured to pause one or more alarms resuscitation/ventilation system 400 is emitting while, for example, a user is reconfiguring resuscitation/ventilation system 400 (e.g., reconnecting a tube or adjusting an operation of resuscitation/ventilation system 400). Airway pressure indicator window 488 may be configured to provide an indication of the air/gas pressure in one or more airways of resuscitation/ventilation system 400 and/or an airway circuit in which resuscitation/ventilation system 400 is a component. High pressure alarm indicator light 490 may be lit, or activated, when a high pressure condition in resuscitation/ventilation system 400 and/or an airway circuit in which resuscitation/ventilation system 400 is a component is connected. Disconnect detected alarm indicator light 492 may be configured to go on when an alarm has been disconnected. Fault alarm indicator light 494 may be configured to go on when a fault alarm is detected. Maximum pressure in centimeters of water (H₂O) dial 496 may be configured to allow a user to adjust a maximum pressure of air/gas resuscitation/ventilation system 400 pushes into an airway circuit for delivery to a patient’s lungs. An exemplary range of maximum pressure is between fifteen (15) and forty (40) cm of H₂O. I/E ratio dial 498 may be configured to allow a user to select between an I/E ratio from 1:1, 1:2, or 1:3. Breaths-per-minute (BPM) dial 499 may be configured to allow a user to adjust a number of breaths per minute resuscitation/ventilation system 400 delivers to the airway circuit. An exemplary range of breaths per minute is from 10-30 breaths per minute. Volume in milliliters (mL) dial 500 may be configured to allow a user to adjust a volume of air delivered by resuscitation/ventilation system 400 to an airway circuit with an exemplary range of volume being between 250-600 mL.

FIG. 4A also shows a second electrical and/or communication coupling portion 441 configured to electrically and communicatively couple with first electrical and/or communication coupling portion 455 via cord 442. In some embodiments, canister 410 may be able to operate physically separate from body 405 (e.g., 2-10 feet away from body 405) and this separation may be facilitated by cord 442 being of sufficient length to accommodate a canister 410 that is physically separate from body 405.

FIG. 4B provides a side plan view of resuscitation/ventilation system 400 and shows canister 410 seated within body 405 and shows a front opening 440 of body 405 through which an upper edge of an indented portion of canister 435, first electrical and/or communication coupling portion 455, and a notch 465 for communication/power cord may be seen. FIG. 4B also provides a side view of a first and a third control panel dials 48 and 50, respectively.

Canister joint 448 may facilitate the seating of canister 410 within body 405 and/or increase the stability of canister 410 when positioned in body 405. Canister 410 may be configured to be removable from body 405 and, in some instances, canister 410 may be released from body 405 and/or locked into place within body 405 via, for example, pushing down and/or pulled up on knob 430. For example, FIG. 4C provides a front plan exploded view of resuscitation/ventilation system 400 where canister 410 is aligned with a canister opening 470 (which is shown in FIG. 4D) and is ready to be inserted into body 405. Alternatively, canister 410 may be extracted from body 405 as shown in FIG. 4C.

In some embodiments, cord 442 is of an extended length as shown in FIG. 4C that, at times, may facilitate use of canister 410 away (e.g., 2-10 feet) from body 405 because cord 442 is configured to provide electrical power to canister 410 as well as instructions for operation (e.g., when and how to move a piston positioned within hollow cylindrical chamber 445 so that air/gas may be pushed out of and/or pulled into hollow cylindrical chamber 445 via extension 450). by extending a reach FIG. 4C also shows cord 442 and second communication/power interface coupling portion 441 where cord 442 is of an extended length that, at times, may facilitate use of canister 410 away from body 405.

FIG. 4D provides a top perspective view of body 405 (i.e., resuscitation/ventilation system 400 with canister 410 removed from body 405) so that a canister orifice 470 is shown. FIG. 4D also provides a stripped-down version of control panel 415 that only provides a first button 41 that corresponds to start/stop button 482, a first indicator light 42 that corresponds to active indicator light 484, a second button 43 that corresponds to pause audio button 486, a display window 44 that corresponds to airway pressure indicator window 488, a second indicator light 45 that corresponds to high pressure alarm indicator light 490, a third indicator light 46 that corresponds to disconnect detected alarm indicator light 492, a fourth indicator light 47 that corresponds to fault alarm indicator light 494, a first dial 48 that corresponds to maximum pressure in centimeters of water H2O dial 496, a second dial 49 that corresponds to I/E ratio dial 498, a third dial 50 that corresponds to breaths-per-minute (BPM) dial 499, and a fourth dial 51 that corresponds to volume in milliliters (mL) dial 500 but does not show the labeling for the various control mechanisms and indicator lists of control panel 415. FIG. 4D also shows a perspective view of notch 465.

FIG. 4E provides a top plan view of body 405. Inside canister orifice 470, a bottom of opening 440, which looks like a shelf may be seen as well as notch 465. In addition, a bottom plate 472 is shown to be the lower surface of base 405 which may be seen through orifice 470. The top plan view of FIG. 4E also shows exemplary components for mounting mechanism 425, which includes a first arm 472, a second arm 474, a joint 474 and a mounting opening 478 and may be configured as a clip. Mounting mechanism 425 may be operated by articulating second arm 474 around joint (by pressing first and second arms 472 and 474 together) so that opening 478 is made larger so that it may fit over, for example, a pole or bracket that may be, for example, extending from a stretcher, bed, or wall and/or resident adjacent to a patient. Then, second arm 474 may be released so that mounting mechanism 425 may close around the pole/bracket so that the pole/bracket is retained within mounting opening 478. FIG. 4D, also provides a side view of first, second, third, and fourth control panel dials 48, 49, 50, and 51, respectively.

FIG. 4F provides a back view of base 405 with canister removed therefrom. The back side of base 405 includes mounting mechanism 425, a power coupling 447, and a pressure line coupling, or pressure port, 457. Power coupling 425 may be configured to connect to a power cord that may be electrically coupled to a main power source. In some cases, the power cord may be coupled to a transformer or wall wort configured to convert AC electrical power to DC electrical power. Pressure port 457 may be configured to couple to a tube that is also coupled to a portion of an airway circuit (e.g., a tap, HMEF, and/or or coupling), such as airway circuit 600. A pressure sensor inside body 405 may register an air/gas pressure within the tube coupled to pressure port 457 and this pressure may be provided to a user or clinician using resuscitation/ventilation system 400 so that, for example, a pressure of the air/gas delivered to the patient may be monitored.

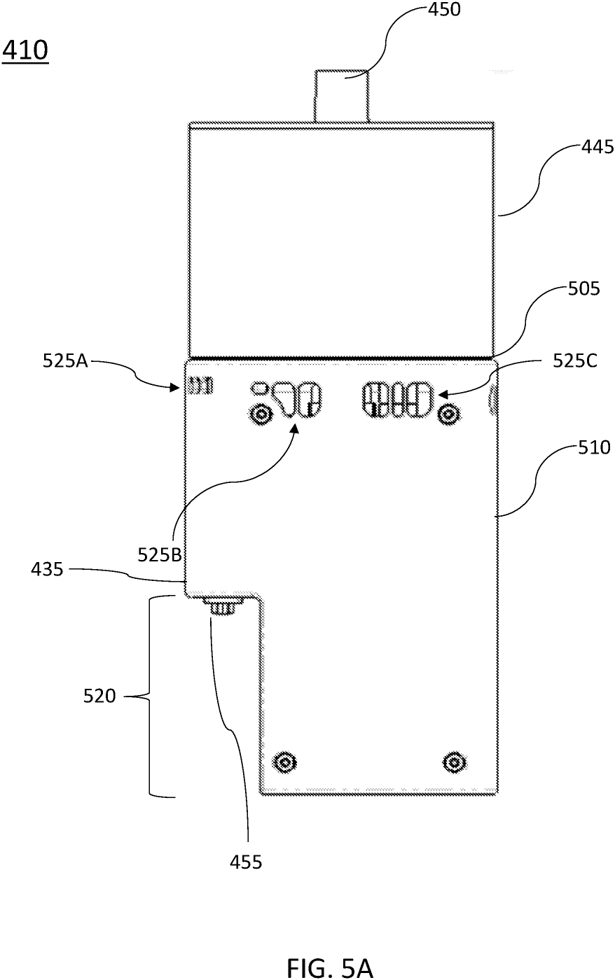

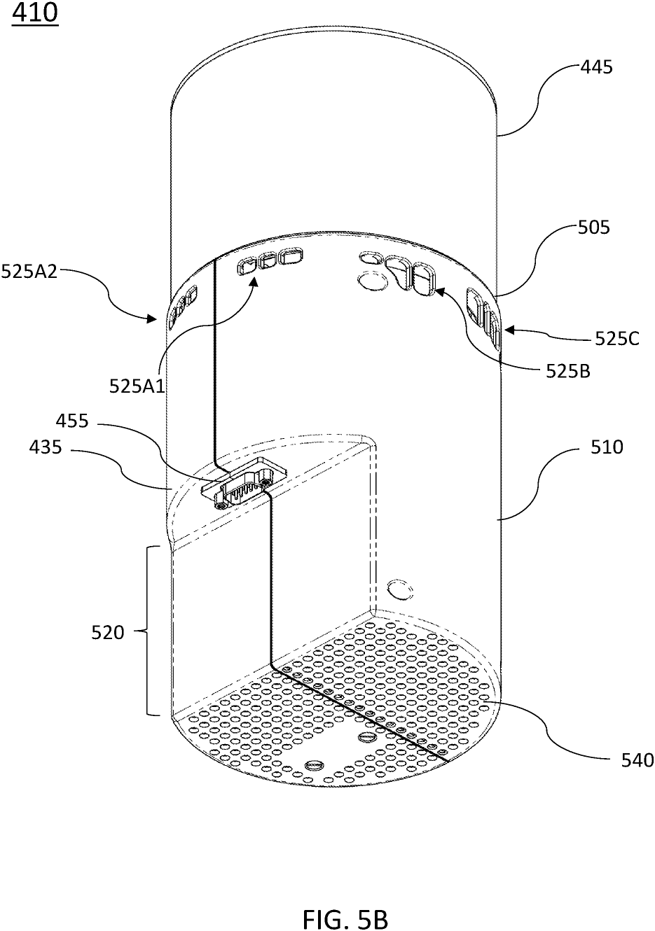

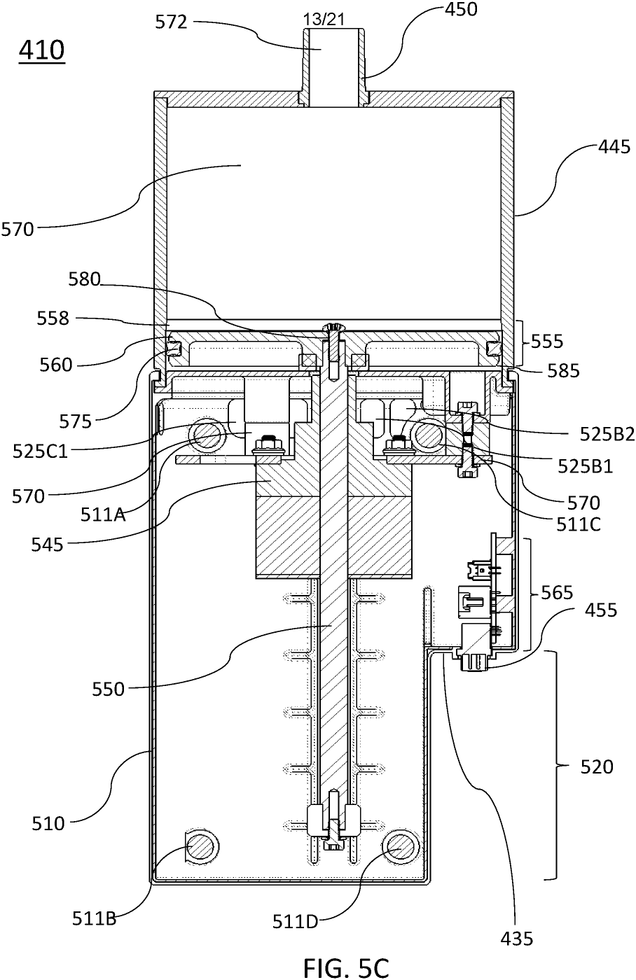

FIG. 5A provides a side view of canister 410 separate and/or removed from body 405. Approximate dimensions for canister 410 are a diameter of 2-8 inches and a height of 4-12 inches. Canister 410 includes hollow cylindrical chamber 445 that terminates at an interface 505, extension 450, and a lower portion 510 configured to be inserted into and be removed from canister orifice 470 of body 405. Lower portion 510 is also configured to reside within body 405 although a flat wall of lower portion 510 may be visible through opening 440. More particularly, lower portion of canister 505 has an indented portion 520 that may be indented approximately 10-40% (e.g., 0.2-3.2 inches) of the diameter of lower portion 510. An upper edge/surface 530 of indented portion 520 includes first electrical and/or communication coupling portion 455.

Lower portion of canister 510 may include a plurality of arrays of ventilation holes including first array of ventilation holes 525A, second array of ventilation holes 525B, and third array of ventilation holes 525C that may be configured to, for example, allow ambient air into canister 410 and/or body 405 for the purpose of, for example, cooling canister 410, body 405, and/or components included therein (e.g., a motor, circuitry, controllers, etc.).

FIG. 5B bottom perspective view of canister 410 that shows a lower perspective view of first communication/power interface coupling portion 455 and a mounting mechanisms 446 for first communication/power interface coupling portion 455. Mounting mechanisms 446 may be configured to accept screws extending from a corresponding second communication/power interface coupling portion 441 attached to cord 442. In addition, FIG. 5B shows that a lower surface 540 of canister 410 includes a plurality of holes that may provide ventilation for one or more components of canister 410.

FIG. 5C provides a cross-section view of canister 410 that shows an inside of one side, or half, of lower portion of canister 510 along with a first, second, third, and fourth attachment mechanism 511A, 511B, 511C, and 511D, respectively that may facilitate the alignment of a first and second half of canister 410 so that they may be affixed together via, for example, a screw, pin, tab, and/or bolt. Also shown in FIG. 5C are a first air/ventilation hole of third array of ventilation holes 525C1 and a first and second air/ventilation hole of second array of ventilation holes 525B1 and 525B2.

FIG. 5C further shows exemplary internal components for canister 410 which include a motor 545, a shaft 550, a piston 555 that includes a piston top layer 558, a piston support member 560, and a piston gasket 575, a printed circuit board (PCB) 565 that includes first electrical and/or communication coupling portion 455 that extends from upper edge of the indented portion of canister 435, a motor mount 570, a chamber, or void, 570 within hollow cylindrical chamber 445, a lumen 572 for extension 450, an attachment mechanism configured to attach piston 555 and piston support member 560 to shaft 550, and a base, or bottom 585 of chamber 445. As shown in FIG. 5C, shaft 550 and piston 555 are in the first position (i.e., the piston is positioned away from extension 550 so that air and/or gas may be allowed into chamber 570).

Motor 545 may be configured to move shaft 550 up and down within canister 410 and this movement of shaft 550 may translate to movement of piston support member 560 and piston 555 within chamber 570 thereby pushing gas out of chamber 570 into lumen 572 and onward through an airway circuit coupled to extension 450 as a combination of shaft 550, piston support member 560, and piston 555 is pushed upwards within chamber 570 and drawing air and/or gas through lumen 572 from an airway circuit coupled to extension 450 as the combination of shaft 550, piston support member 560, and piston 555 is pulled downwards toward a base of chamber 570.

Piston gasket 575 may be configured to encircle piston support member 560 as, for example, a ring and may be made from a have a deformable material (e.g., latex, vinyl, or rubber) that facilitates an air-tight, or nearly air-tight, seal with an interior wall of chamber 570 as piston 555 articulates between a first position (as shown in FIG. 5C) to the second position (i.e., piston 555 and piston support member 560 are positioned closer to the top of chamber 570 than what is shown in FIG. 5C). Additionally, or alternatively, piston top layer 558 may be configured to have a circumference that matches, or substantially matches a circumference of chamber 570 so that piston top layer 558 achieves an air-tight, or substantially air-tight, seal with chamber 570.

PCB 565 may include one or more components that facilitate communication with base 405 including a pinned connector and circuitry that communicates instructions to motor 545. Exemplary instructions communicated to motor 545 include, but are not limited to on/off, speed of motion, a duration/how long to push piston 555 upward, and/or downward within chamber 570 when translating from the first position and second position.

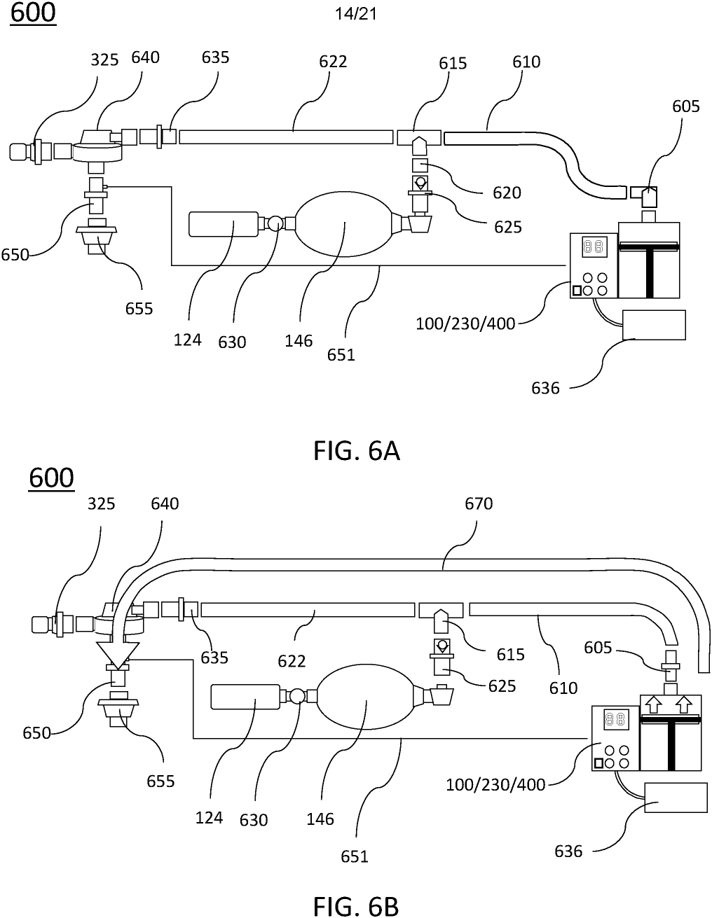

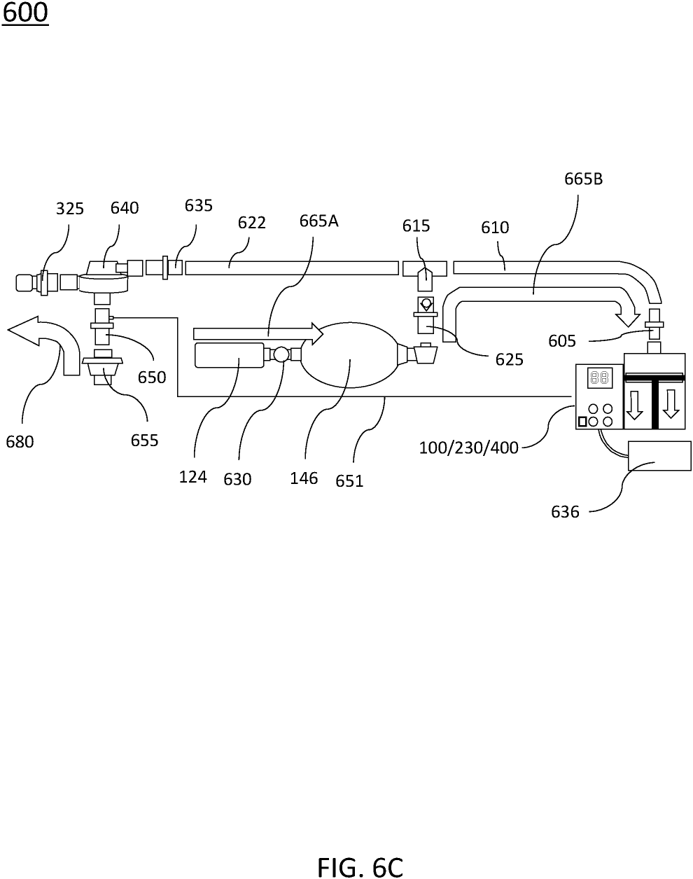

FIGS. 6A-6C provide schematic diagrams of an exemplary airway circuit 600 including exemplary piston resuscitator and/or ventilator system like piston resuscitator and/or ventilator system 230, 100, or 400 that may be used to provide artificial respiration and/or ventilation to a patient. Resuscitator and/or ventilator system 230, 100, or 400 may draw power from an electrical main via an AC/DC transformer/wall wort 636. As shown in FIG. 6A, airway circuit 600 includes a like piston resuscitator and/or ventilator system 100, 230, 400 coupled to a coupling 605, which is coupled to a first tube 610. First tube 610 is coupled to a Y-connector 615, which couples to a second tube 620. Second tube 620 couples to a check valve 625, which couples to ventilator bag 146, which is coupled to a coupling 630, which is coupled to air/gas/oxygen reservoir bag 124. Y-connector 615 also couples to a third tube 622, which is coupled to a connector 635. Connector 635 is coupled to a non-rebreathing valve (NRV) 640, which is coupled to a PEEP valve 325 and a tap 650. Tap 650 is coupled to a heat and moisture exchange filter (HMEF) 655, which may be coupled to a patient airway mechanism such as patient mask 265. NRV 640 may be any valve configured as a one-way valve (or, in some instances, a check valve) to allow the expulsion of air/gas from airway circuit 600 while preventing air or other gasses from entering airway circuit. HMEF 655 may be configured to retain a patient’s own humidity and moisture during the exhalation process within airway circuit so that, for example, air/gas delivered to the patient on inhalation is not too dry, which can cause damage to sensitive tissues along the patient’s respiratory tract. HMEF 665 may also filter particles (e.g., bacteria, viruses, etc.) from air exhaled by the patient. Additionally, tap 650 is coupled to a first end of a pressure line tube 651, which may be configured as a simple flexible tube with a lumen running along the length of the pressure line tube 651. A second end of pressure line tube 651 may be coupled to a pressure port like pressure port 457. An air/gas pressure within pressure line tube 651 may be measured by, for example, a pressure sensor resident within resuscitation/ventilation system 400.

FIG. 6B shows a first air pathway 670 through airway circuit 600 when a patient is inhaling and/or air/gas is being pushed into the patient’s lungs. At first, a piston like piston 105, 350, and/or 555 is pushed upward from the first position to the second position within a cylinder and/or chamber like cylinder and/or chamber 112, 330, and/or 570. This upward movement of the piston acts to compress the air/gas positioned within the chamber and force the air/gas out of the chamber and into coupling 605 so that the air/gas may be pushed through airway circuit 600 from coupling 605, through first tube 610, Y-connector 615, second tube 620, connector 635, NRV 640, tap 650, and into a patient airway (e.g., a mask).

FIG. 6C shows a second air pathway 655 and a third air pathway 680 through airway circuit 600 when a patient is exhaling and/or air/gas is being exhaled out from the patient’s lungs. At first, a piston is pulled downward from the first position to the second position within the cylinder and/or chamber. This creates a negative pressure in airway circuit 600 which draws air/gas from air/gas/oxygen reservoir bag 124 into ventilator bag 146 via a first portion of a first portion of second air pathway 665A. A second portion of second air pathway 665B is pulled from ventilator bag 146, through check valve 625, and into the cylinder/chamber typically until the piston stops moving from the second position to the first position. In addition, FIG. 6C shows a third air flow 680 of air/gas that is exhaled by the patient and is pushed out through HMEF 660, tap 650, and PEEP 625 into the ambient air.

FIG. 7 provides an example of a system 700 that may be representative of a computing and/or processing system that may be configured to control the operation of a resuscitator and/or ventilator system like the resuscitator and/or ventilator systems described herein (e.g., system 100, 230, and/or 400) and/or components thereof and/or execute one or more of the processes disclosed herein. System 700 may be resident in a control panel like control panel 170 or in a control/piston system like control/piston system 230. In the example of resuscitation/ventilation system 400, system 700 may reside primarily in body 405. Note, not all of the resuscitation/ventilation system disclosed herein have all of the features of system 700.

System 700 includes a bus 702 or other communication mechanism for communicating information, and a processor 704 coupled with the bus 702 for processing information and/or providing instructions to one or more components of system 700. System 700 also includes a main memory 706, such as a random access memory (RAM) or other dynamic storage device, coupled to the bus 702 for storing information and instructions to be executed by processor 704. Main memory 706 also may be used for storing temporary variables or other intermediate information during execution of instructions to be executed by processor 704. System 700 further includes a read only memory (ROM) 708 or other static storage device coupled to the bus 702 for storing static information and instructions for the processor 704. A storage device 710, for example a hard disk, flash memory-based storage medium, or other storage medium from which processor 704 can read, is provided and coupled to the bus 702 for storing information and instructions (e.g., operating systems, applications programs, and the like).

System 700 may be coupled via the bus 702 to a display 705, such as a flat panel display, for displaying information to a computer user. In some embodiments, system 700 may be implemented as an airway pressure indicator window such as airway pressure indicator window 488.

Input device 710 may include any device by which a user may enter information into system 700. Input device 710 may be coupled to the bus 702 for communicating information, command selections, directional information, gestures, and controlling cursor movement of/input by the user to the processor 704. Exemplary input devices 710 include, but are not limited to, a keyboard including alphanumeric and other keys, mouse, track pad, a touch screen, a button such as start/stop button 482 and/or pause audio button 486 and/or a dial such as maximum pressure in centimeters of water H2O dial 496, I/E ratio dial 498, breaths-per-minute (BPM) dial 499, and volume in milliliters (mL) dial 500.

System 700 may include one or more indicator lights 722. Exemplary indicator lights 722 include, but are not limited to, an active indicator light like active indicator light 484, a high pressure alarm indicator light like high pressure alarm indicator light 490, a disconnect detected alarm indicator light like disconnect detected alarm indicator light 492, and/or a fault alarm indicator light like fault alarm indicator light 494. Activation of one or more indicator lights 722 may be triggered by, for example, an instruction from processor 704, which may be responsive to information received via communication interface 718 and/or input device 710 from, for example, a pressure sensor or other sensor coupled to system 700.

System 700 may further include an antenna 720 configured to broadcast and/or receive signals to/from, for example, an external computer (e.g., an external control that may be operated via a software application running on the external computer) panel and/or components of ventilation system 100. These signals may be, for example, updates to software and/or commands to one or more components of system 700 and/or the resuscitation/ventilation system disclosed herein.

The processes referred to herein may be implemented by processor 704 executing appropriate sequences of computer-readable instructions contained in main memory 706. Such instructions may be read into main memory 706 from another computer-readable medium, such as storage device 710, and execution of the sequences of instructions contained in the main memory 706 causes the processor 704 to perform the associated actions. In alternative embodiments, hardwired circuitry or firmware-controlled processing units may be used in place of, or in combination with, processor 704 and its associated computer software instructions to implement the invention. The computer-readable instructions may be rendered in any computer language.

In general, all of the process descriptions provided herein are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as system 700 or similar electronic computing device, that manipulates and transforms data represented as physical (electronic) quantities within its registers and memories into other data similarly represented as physical quantities within its memories or registers or other such information storage, transmission or display devices.

System 700 also includes a motor driver 716 configured to drive, or otherwise operate, a motor such as motor 360 and/or motor 545. In some cases, motor driver 716 may operate responsively to instructions from processor 704.

System 700 also includes a communication interface 718 coupled to the bus 702. Communication interface 718 may provide a two-way data communication channel with a computer network, which provides connectivity to and among the various computer systems discussed above. For example, communication interface 718 may be a local area network (LAN) card to provide a data communication connection to a compatible LAN, which itself is communicatively coupled to the Internet through one or more Internet service provider networks. The precise details of such communication paths are not critical to the present invention. What is important is that system 700 can send and receive messages and data through the communication interface 718 and, in that way, communicate with hosts accessible via the Internet. It is noted that the components of system 700 may be located in a single device or located in a plurality of physically and/or geographically distributed devices.

System 700 also includes a power source 724 configured to provide power to one or more components of system 700 and/or the resuscitation/ventilation system described herein. Exemplary power sources include, but are not limited to, batteries, rechargeable batteries, and a port by which to connect to an electrical main via a power cord.

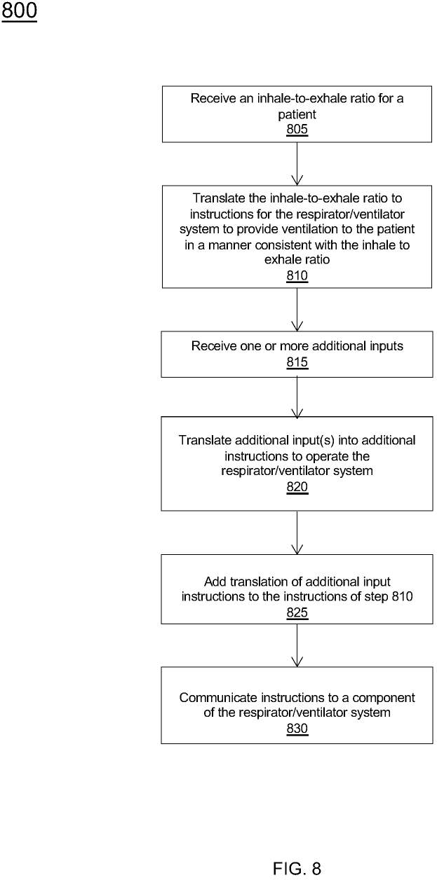

FIG. 8 is a flowchart showing a process 800 for providing instructions to a ventilator for the provision ventilation to a patient. process 800 may be executed by a processor like processor 704 communicatively coupled to a resuscitation/ventilation system like resuscitation/ventilation system 100, 200, and/or 400.

In step 805, an inhale-to-exhale (I/E), or breathing, ratio for a patient using a resuscitator and/or ventilator system and/or an airway circuit like those described herein may be received. The inhale-to-exhale ratio may be determined by a clinician responsively to an examination of the patient. If the patient is having trouble oxygenating his or her blood, the inhale-to-exhale ratio may be set to 1:1 so that a duration of time for pushing air out of the resuscitator and/or ventilator system into the patient’s lungs is equal, or substantially similar to, a duration of time for pulling air from the patient’s lungs, or allowing the patient to exhale, thereby assisting exhalation. An inhale-to-exhale ratio of 1:1 would be similar to a patient taking frequent short and shallow breaths.

If the patient is having trouble removing carbon dioxide from his or her lungs, the inhale-to-exhale ratio may be set to 1:3 or 1:4 so that a duration of time for pushing air out of the resuscitator and/or ventilator system into the patient’s lungs is shorter (e.g., 8 s) than a duration of time for pulling air from the patient’s lungs (e.g., 6 s) and/or waiting for the patient to exhale thereby assisting exhalation. An inhale-to-exhale ratio of 1:3 or 1:4 would be similar to a patient taking long deep breaths with a long exhalation cycle so as to assist with, for example, the exhalation of carbon dioxide from the patient’s lungs.

In step 810, the inhale-to-exhale ratio for the patient may be translated into a set of instructions for operating a resuscitator and/or ventilation system like resuscitation/ventilation system 100, 200, and/or 400 and/or a resuscitation/ventilation system like resuscitation/ventilation system 110, 230, and/or 400. In some embodiments, when system 100 is used, execution of step 810 may include setting a pressure level for pressure chamber 112 for the first and the second states of pressure chamber 112 and a time cycle for achieving a low pressure state and a high pressure state and/or cycling between the low and high pressure states for pressure chamber 112. In some embodiments, the set of instructions may include instructions to open and/or close pressurized air valve 122, which may be communicatively or electronically coupled to the processor so that pressurized air valve 122 opens responsively to an instruction to do so that pressurized air may flow into pressure chamber to increase the air pressure in pressure chamber. In other embodiments, when pressurized air valve 122 is open at all times, the instruction may be to activate (i.e., turn on) an air compressor, or pump, so that it forces compressed air through pressurized air valve 122 thereby filling pressure chamber 112 with air and creating a high pressure in pressure chamber 112. Additionally, or alternatively, the set of instructions may include an instruction to turn a suction pump (which may be the same as the air compressor/pump that forces compressed air through pressurized air valve 122 - just operating in a reverse method) so that the suction pump evacuates air from pressure chamber 112 through pressurized air valve 122 thereby emptying pressure chamber 112 of most, if not all, of the air contained therein and creating a low pressure in pressure chamber 112. In some embodiments, the instructions may also include an air flow rate by which the pressure chamber 112 should be filled with compressed air and/or evacuated of air by an air pump.

In embodiments where system 230 or 400 are used, execution of step 810 may include translating the received I/E ratio into instructions for a motor, such as motor 360 or 545, respectively, regarding setting a time cycle and/or a velocity for translating a piston, such as piston 350 or 555 and/or a shaft connected to a piston such as shaft 345 or 550, respectively, from the first state to the second state and translating the piston and/or shaft from the second state to the first state. In some embodiments, setting the time cycle may include instructions for when to start and/or stop motion and/or duration of time for the movement of piston shaft from the first position to the second position and from the second position to the first position. In some embodiments setting a velocity for translating the piston and/or shaft may include setting one or more acceleration rates and/or velocities (e.g., a constant velocity) for piston and/or shaft and/or a duration of time for the application of force to the piston and/or shaft to cause the respective acceleration and/or movement of the piston and/or shaft at the desired velocity.

Optionally, in step 815, additional inputs for one or more additional settings for the operation of the resuscitation/ventilation system may be received. Additional inputs for settings include, but are not limited to a setting for maximum pressure (e.g., 15-40 centimeters of water (cm H₂O)), a number of breaths per minute (e.g., 10-30), and/or a volume of air/gas (e.g., 250-600 mL) to be delivered to the patient’s lungs and/or pushed through the resuscitation/ventilation system and/or airway circuit. The additional inputs/settings may be translated into additional instructions for operating the resuscitation/ventilation system (step 820) and added into the set of instructions initially generated in step 810 (step 825). Then, the instructions from step 810 and/or 825 may be communicated to the RESUSCITATION/VENTILATION SYSTEM and/or a component thereof (step 830) so that artificial respiration and/or ventilation may be provided to a patient coupled to the resuscitation/ventilation system in a manner consistent with the received inhale-to-exhale ratio and/or additional inputs.

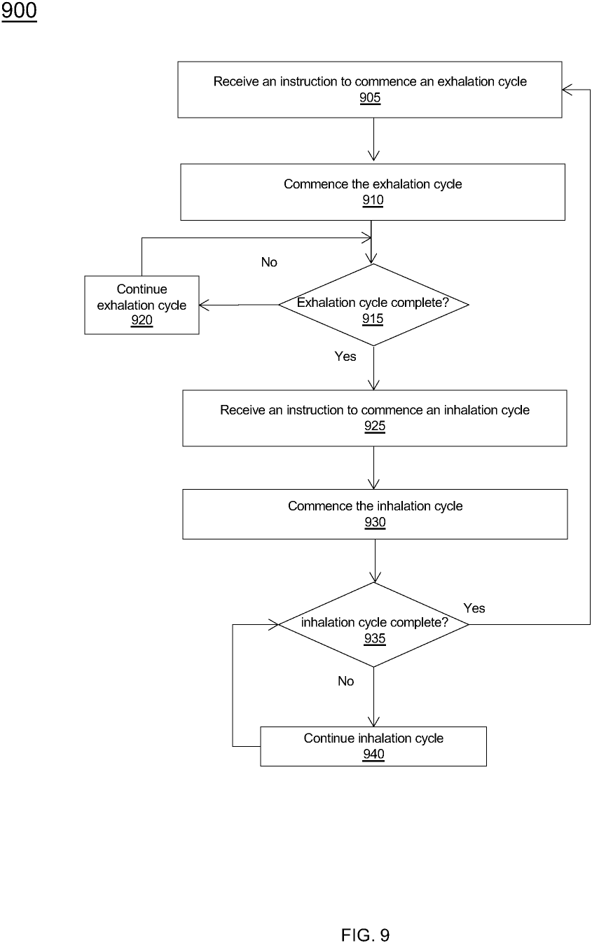

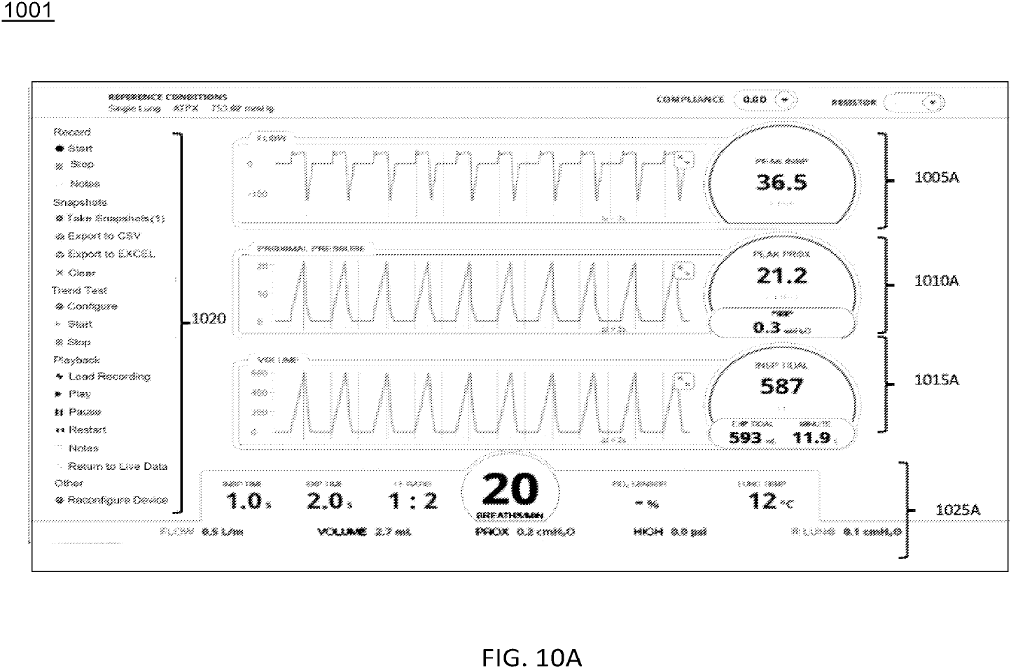

FIG. 9 is a flowchart showing a process 900 for executing instructions to operate a resuscitation/ventilation system for the provision ventilation and/or artificial respiration to a patient. In some embodiments, execution of process 900 is an example of how a set of instructions that may be communicated to a resuscitation/ventilation system in step 830 of process 800 may be executed by the resuscitation/ventilation system and/or a component thereof. process 900 may be executed by a resuscitation/ventilation system like resuscitation/ventilation system 100, 200, and/or 400 and/or a processor like processor 704 communicatively coupled to a resuscitation/ventilation system like resuscitation/ventilation system 100, 200, and/or 400.

In step 905, an instruction to commence an exhalation cycle for a patient receiving ventilation from the resuscitator and/or ventilator system may be received and, in step 910, the exhalation cycle may be initiated. The exhalation cycle may be commenced by moving a piston like piston 105, 350, and/or 555 from a second position (i.e., at the bottom of a chamber like chamber 112 and/or 570 as shown in FIGS. 1B and 5C, respectively) to a first position (i.e., to the top of a chamber like chamber 112, 330, and/or 570) and thereby drawing air/gas into a pressure chamber like chamber 112, 330, and/or 570 from an airway circuit like the airway circuits explained above with regard to FIGS. 1A, 1B, 2, 3, and 6A-6C.

In step 915, it may be determined if the exhalation cycle is complete and, if not, the exhalation cycle may continue (step 920). A determination of whether the exhalation cycle is complete may involve a determination of whether a duration of time for an exhalation cycle has expired and/or whether an air pressure within the system indicates that the patient has exhaled. When the exhalation cycle is complete, an instruction to commence an inhalation cycle may be received (step 925).

In step 930, the inhalation cycle may be commenced by moving a piston like piston 105, 350, and/or 555 from a first position (i.e., at the top of a chamber like chamber 112 and/or 570 as shown in FIG. 1A) to a second position (i.e., to the bottom of a chamber like chamber 112, 330, and/or 570) and thereby pushing air/gas from pressure chamber like chamber 112, 330, and/or 570 into the airway circuit like the airway circuits explained above with regard to FIGS. 1A, 1B, 2, 3, and 6A-6C.

A determination of whether the inhalation cycle is complete may then be made (step 935) and if not, the inhalation cycle may be continued (step 940). A determination of whether the inhalation cycle is complete may involve a determination of whether a duration of time for an inhalation cycle has expired and/or whether an air pressure within the system indicates that the patient has fully inhaled. When the inhalation cycle is complete, process 900 may proceed to step 905 and process 900 may be repeated.

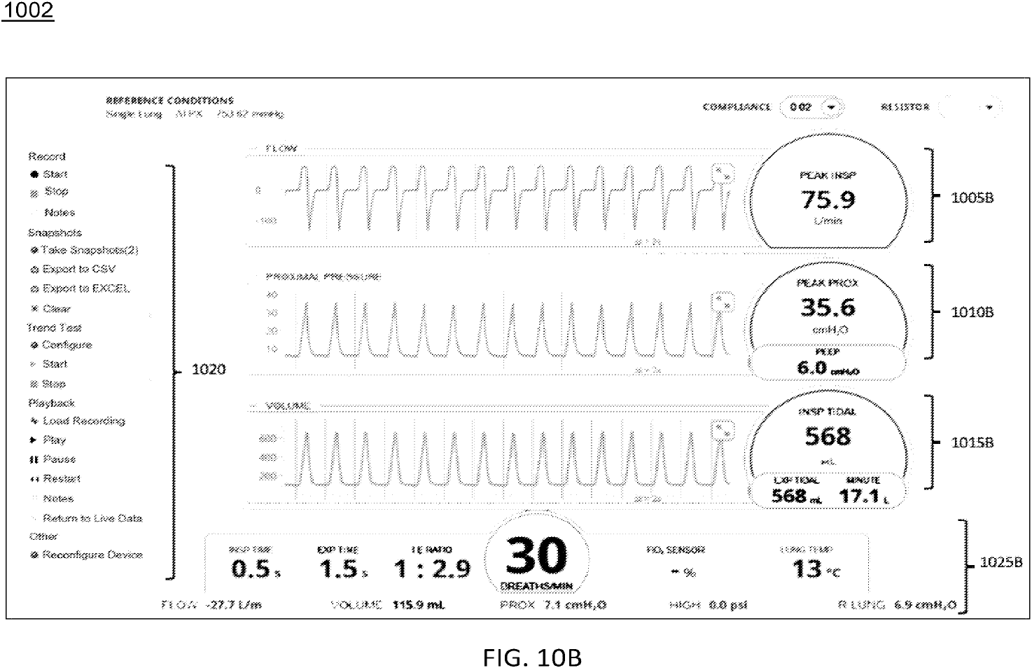

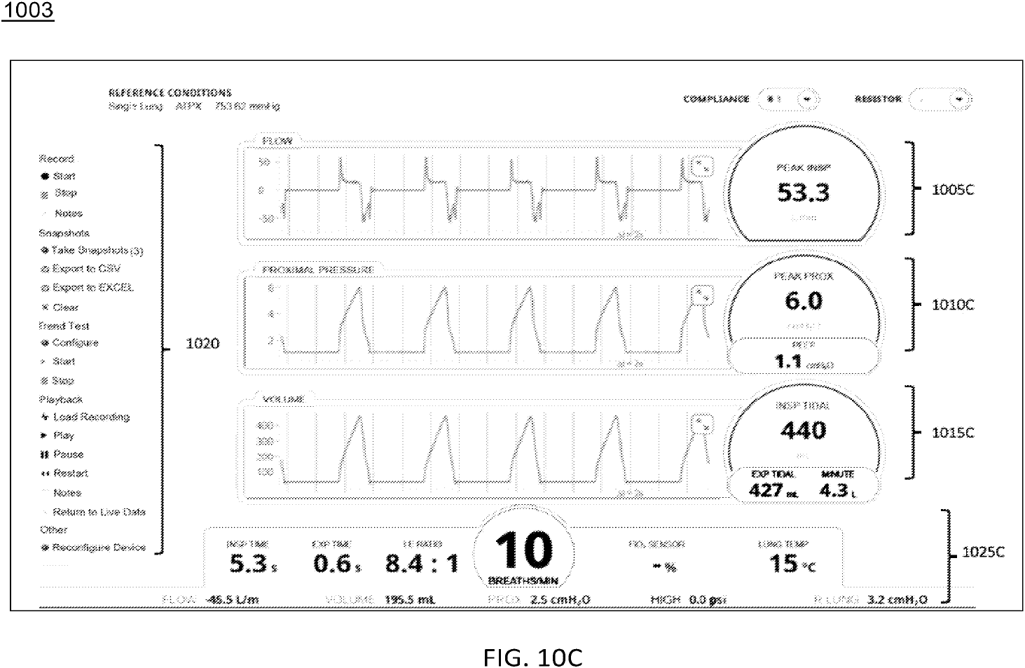

FIGS. 10A, 10B, and 10C provide a first graphic user interface (GUI) 1001, a second GUI 1002, and a third GUI 1003, respectively, all of which show various settings and performance metrics for a resuscitation/ventilation system like resuscitation/ventilation system 100, 200, 400, and/or components thereof. The settings include an inspiratory time in seconds, an expiratory time in seconds, an I/E ratio, a number of breaths per minute, a sensor value, a lung temperature in degrees Celsius, a flow rate in L/m, a volume of air/gas delivered in mL, a proximal pressure value, and a lung pressure. One or more values or operating conditions for resuscitation/ventilation system 100, 200, 400, and/or components thereof may be set via selecting one or more commands and/or options provided by a menu 1020 that provides commands allowing a user/operator to record an operation of the resuscitation/ventilation system 100, 200, 400, and/or components thereof, start and/or stop operation of resuscitation/ventilation system 100, 200, 400, and/or components thereof, take a snapshot of a GUI like GUIs 1001, 1002, and/or 1003 and/or data provided thereon. In addition, menu 1020 provides a user/operator with the option to export values displayed on a GUI like GUIs 1001, 1002, and/or 1003 in a comma separated values (CSV) and/or MICROSOFT EXCEL™ format. Menu 1020 also provides a user/operator with commands that allow the user to configure, start, and/or stop a trend test, load a recording, playback pause, stop, and/or add notes to data and/or a loaded recording. Finally, menu 1020 also provides icons, the selection of which enables a user to return to live/real time data generated by resuscitation/ventilation system 100, 200, 400, and/or components thereof and/or reconfigure resuscitation/ventilation system 100, 200, 400, and/or components thereof.

The performance metrics are shown with three graphic elements, where a first graphic element 1005 includes a flow rate over time, and a peak inspiratory volume in liters/minute, a second graphic element 1010 shows proximal pressure (in cmH₂0) delivered to the patient over time, a peak proximal pressure (in cmH₂O) delivered to the patient, a PEEP value in cmH₂O, a third graphic element 1015 shows a tidal volume in mL, an inspiratory tidal value, an expiratory tidal value, and a total volume delivered over a minute, and a statistics bar 1025 that provides various statistics regarding performance of and/or settings for resuscitation/ventilation system 100, 200, 400, and/or components thereof. More particularly, statistics bar 1025 provides a value for inspiratory time (shown as insp time on GUIs 1001, 1002, and 1003), expiratory time (shown as exp time on GUIs 1001, 1002, and 1003), I:E ratio, a count of breaths per minute, a value provided by an FiO₂ sensor, a lung temperature, a flow rate, a volume of gas delivered, a proximity value, a high pressure value, and a lung pressure value.

First GUI 1001 of FIG. 10A provides performance metrics for the resuscitation/ventilation system when a first set of settings and/or inputs are received at, for example, step(s) 805 and 815 via, for example, menu 1020 and/or a control panel like control panel 415. In the example of GUI 1001, the PEEP valve value is set very low, in this case 0.3 cmH₂O, the breaths per minute is set to 20 BPM, the I/E ratio is set at 1:2, and the volume is set at 600 mL. The corresponding performance metrics of first graphic element 1005A show how the flow of air/gas changes over a time interval of 2 seconds and also a peak inspiratory value of 36.5 L/minute over the 2 second time interval. The corresponding performance metrics of second graphic element 1010A show how the proximal pressure of air/gas delivered to a patient and/or lung changes over the 2 second time interval and also a peak proximal pressure value of 21.2 cmH₂O and a PEEP value of 0.3 cmH₂O over the 2 second time interval. The corresponding performance metrics of third graphic element 1015A show how the volume of air/gas delivered to a patient and/or lung changes over the 2 second time interval and also an inspiratory tidal volume value of 587 mL, an expiratory tidal volume of 593 mL, and a volume of 11.9 L per minute. The corresponding statistics for statistics bar 1025A provide an inspiratory time of 1 s, expiratory time of 2 s, an I:E ratio of 1:2, a count of breaths per minute of 20 bpm, no value provided by an FiO₂ sensor, a lung temperature of 12° C., a flow rate of 0.5 L/m, a volume of gas delivered of 2.7 mL, a proximity pressure value of 0.2 cmH₂O, a high pressure value of 0.0 pounds per square inch (PSI), and a lung pressure value of 0.1 cmH₂O.

Second GUI 1002 of FIG. 10B provides performance metrics for the resuscitation/ventilation system when a second set of settings and/or inputs are received at, for example, step(s) 805 and 815. In the example of GUI 1002, the PEEP valve value is set to 6 cmH₂O, the breaths per minute is set to 30 BPM, the I/E ratio is set at 1:3, and the volume is set at 600 mL. The corresponding performance metrics of first graphic element 1005B show how the flow of air/gas changes over a time interval of 2 seconds and also a peak inspiratory value of 75.9 L/minute over the 2 second time interval. The corresponding performance metrics of second graphic element 1010B show how the proximal pressure of air/gas delivered to a patient and/or lung changes over the 2 second time interval and also a peak proximal pressure value of 35.6 cmH₂O and a PEEP value of 6 cmH₂O over the 2 second time interval. The corresponding performance metrics of third graphic element 1015B show how the volume of air/gas delivered to a patient and/or lung changes over the 2 second time interval and also an inspiratory tidal volume value of 568 mL, an expiratory tidal volume of 568 mL, and a volume of 17.1 L per minute. The corresponding statistics for statistics bar 1025B provide an inspiratory time of 0.5 s, expiratory time of 1.5 s, an I:E ratio of 1:2.9, a count of breaths per minute of 30 bpm, no value provided by an FiO₂ sensor, a lung temperature of 13° C., a flow rate of -27.7 L/m, a volume of gas delivered of 115.9 mL, a proximity pressure value of 7.1 cmH₂O, a high pressure value of 0.0 pounds per square inch (PSI), and a lung pressure value of 6.9 cmH₂O.

Third GUI 1003 of FIG. 10C provides performance metrics for the resuscitation/ventilation system when a third set of settings and/or inputs are received at, for example, step(s) 805 and 815. In the example of GUI 1003, the PEEP valve value is set to 1.1 cmH₂O, the breaths per minute is set to 10 BPM and the I/E ratio is set at 8.4/1. The corresponding performance metrics of first graphic element 1005C show how the flow of air/gas changes over a time interval of 2 seconds and also a peak inspiratory value of 53.3 L/minute over the 2 second time interval. The corresponding performance metrics of second graphic element 1010C show how the proximal pressure of air/gas delivered to a patient and/or lung changes over the 2 second time interval and also a peak proximal pressure value of 6 cmH₂O and a PEEP value of 1.1 cmH₂O over the 2 second time interval. The corresponding performance metrics of third graphic element 1015C show how the volume of air/gas delivered to a patient and/or lung changes over the 2 second time interval and also an inspiratory tidal volume value of 440 mL, an expiratory tidal volume of 427 mL, and a volume of 4.3 L per minute. The corresponding statistics for statistics bar 1025C provide an inspiratory time of 5.3 s, expiratory time of 0.6 s, an I:E ratio of 8.4:1, a count of breaths per minute of 10 bpm, no value provided by an FiO₂ sensor, a lung temperature of 15° C., a flow rate of -45.5 L/m, a volume of gas delivered of 195.5 mL, a proximity pressure value of 2.5 cmH₂O, a high pressure value of 0.0 pounds per square inch (PSI), and a lung pressure value of 3.2 cmH₂O.

In some embodiments, one or more of the resuscitator and/or ventilator systems described herein may include an air/gas intake filter and/or an air/gas exhaust filter positioned near corresponding air/gas intake and/or exhaust ports/couplings. The filters may filter for pathogens (e.g., bacteria or virus), particles (dust, etc.) or other foreign or undesirable particles.

In some embodiments, one or more of the resuscitator and/or ventilator systems described herein may include one or more pressure sensors. At times, one or more of the pressure sensors may be positioned near the patient interface like an endotracheal tube. Pressure sensor readings may be used to, for example, control the operation of the resuscitator and/or ventilator system which may include triggering the inspiration cycle based on a determination (made in conjunction with pressure sensor information) that the patient is attempting to breathe spontaneously. Additionally, or alternatively, one or more of the resuscitator and/or ventilator systems described herein may include one or more mechanisms to regulate pressure within the resuscitator and/or ventilator system or a portion thereof. These pressure regulation mechanisms may include a PEEP valve, a pop-off valve, and/or a pressure release valve.

Resuscitator and/or ventilator systems described herein may be powered by, for example, a battery and/or a power cord configured to be coupled to a standard wall outlet.

We claim:

1. A system comprising:
a canister configured to be removably inserted into an opening in a body, the canister comprising:
a hollow cylindrical chamber;
a piston sized and shaped to fit within the hollow cylindrical chamber;
an opening in the hollow cylindrical chamber through which gas may pass into a respiratory circuit for a patient as the piston translates between a first position within the chamber and a second position within the chamber;
a motor configured to move the piston from a first position to a second position and subsequently move the piston from the second position to the first position within the hollow cylindrical chamber;
a first communication/power interface coupling portion configured to couple to a corresponding second communication/power interface coupling portion and establish communicative and electrical coupling between the canister and the body;
the body, the body comprising:
the opening configured for acceptance of a portion of the canister therein;
a controller configured to control the motor;
the second communication/power interface coupling portion configured to couple to the first communication/power interface coupling portion and establish communicative and electrical coupling between the canister and the body; and
a power source configured to provide electrical power to the body and the cannister.

2. The system of claim 1, wherein the motor is a stepper motor.

3. The system of claim 1, wherein the canister is configured to be replaceable with another canister.

4. The system of claim 1, wherein the canister further comprises:
a shaft in mechanical communication with the piston and the motor.

5. The system of claim 4, wherein the motor is configured to move the piston between the first position and the second position via moving the shaft up and down.

6. The system of any of claims 1-5, wherein the body further comprises:
an engagement mechanism configured to enable removal of the canister from the body.

7. The system of any of claims 1-6, wherein the body further comprises:
a control panel, the control panel being configured to accept user inputs for setting at least one of a maximum pressure, an inhale to exhale (I/E) ratio, a number of breaths per minute, and a volume of gas delivered to a patient.

8. The system of any of claims 1-7, wherein the body further comprises:
a motor driver communicatively coupled to the controller and configured to provide instructions to the motor that cause the motor to move the piston between the first position and the second position and between the second position and the first position.

9. The system of any of claims 1-8, wherein the canister is configured to reside outside the body.

10. The system of claim 9, wherein the canister is configured to operate when the canister is positioned outside of the body via at least one of a remote communicative coupling and a power coupling to the body.

11. The system of any of claims 1-10, wherein the second communication/power interface coupling portion is coupled to a cord.

12. The system of claim 11, wherein a length of the cord is in a range of 1-10 feet.

13. The system of claim 11, wherein the first and second communication/power interface coupling portions are configured to communicate wirelessly.

14. The system of any of claims 1-13, wherein the canister further comprises a battery.

15. An airway circuit comprising:
an air source configured to draw in a gas;
a connector, the connector coupling the air source to a system and being configured to allow the gas to flow therethrough
the resuscitation/ventilation system, the system comprising:
a canister configured to be removably resident within an opening in a body, the canister comprising:
a hollow cylindrical chamber;
a piston sized and shaped to fit within the hollow cylindrical chamber;
an opening in the hollow cylindrical chamber through which gas may pass into a respiratory circuit for a patient as the piston translates between a first position within the chamber and a second position within the chamber;
a motor configured to move the piston from a first position to a second position and subsequently move the piston from the second position to the first position within the hollow cylindrical chamber;
a first communication/power interface coupling portion configured to couple to a corresponding second communication/power interface coupling portion and establish communicative and electrical coupling between the canister and the body;
the body, the body comprising:
the opening configured for acceptance of a portion of the canister therein;
a controller configured to control the motor;
the second communication/power interface coupling portion configured to couple to the first communication/power interface coupling portion and establish communicative and electrical coupling between the canister and the body; and
a power source configured to provide electrical power to the body and the cannister; and
a tube coupled to the connector and a patient airway mechanism; the tube being configured to deliver the gas from the system to the patient airway mechanism.

16. The airway circuit of claim 15, further comprising:
a positive end-expiratory pressure (PEEP) valve.
